(19) United States
(12) Patent Application Publication
(10) Pub. No.: US 2025/0302933 A1
(43) Pub. Date: Oct. 2, 2025

(54) ATTENUATED YET IMMUNOGENICALLY POTENTIATED TUMOR EXTRACELLULAR VESICLE COMPOSITIONS AND USES THEREOF

(71) Applicant: KOREA INSTITUTE OF SCIENCE AND TECHNOLOGY, Seoul (KR)

(72) Inventors: In-San KIM, Seoul (KR); Jihoon HAN, Seoul (KR); Seohyun KIM, Seoul (KR)

(73) Assignee: KOREA INSTITUTE OF SCIENCE AND TECHNOLOGY, Seoul (KR)

(21) Appl. No.: 19/097,584

(22) Filed: Apr. 1, 2025

(30) Foreign Application Priority Data
Apr. 2, 2024 (KR) 10-2024-0044720

(51) Int. Cl. A61K 39/00; A61P 35/00; A61P 37/04; C12N 5/00; C12N 5/09

(52) U.S. Cl. CPC A61K 39/0011; A61P 35/00; A61P 37/04; C12N 5/0018; C12N 5/0693; A61K 2039/55555; C12N 2501/999

(57) ABSTRACT

The present disclosure relates to attenuated yet immunogenically potentiated tumor-derived extracellular vesicle compositions and uses thereof. The present disclosure was completed by confirming that the extracellular vesicles secreted by tumor cells are attenuated yet immunogenically potentiated through a process of treating tumor cells with verteporfin, and the extracellular vesicles of the present disclosure can be used as a vaccine composition, etc.

### CROSS-REFERENCE TO RELATED APPLICATION

This application claims the benefit of Korean Patent Application No. 10-2024-0044720 filed on Apr. 2, 2024, in the Korean Intellectual Property Office, the entire disclosure of which is incorporated herein by reference for all purposes.

### BACKGROUND

### 1. Field of the Invention

The present disclosure relates to attenuated yet immunogenically potentiated tumor extracellular vesicle compositions, vaccine compositions including the same as active ingredients, and the like.

### 2. Description of the Related Art

Despite recent developments in surgical techniques and new cancer treatment strategies, the risk of cancer recurrence remains high enough to pose a threat to numerous patients. Customized cancer vaccination is known as one of the most promising strategies for inducing and amplifying individually designed tumor-specific immune responses using various antigens produced directly from a patient's tumor tissue. However, in order to develop a successful cancer vaccine, there is a need to meet several requirements, such as an optimal tumor antigen source, a combination with an immune adjuvant, and the like, and all of these requirements have a technical challenge to be formulated in an appropriate delivery platform. Many efforts have been made over the past several decades to develop customized cancer vaccines, but most of the efforts have failed to meet these factors and have shown unsatisfactory clinical results. Tumor extracellular vesicles (TEVs) are lipid bilayer particles released from tumor cells into an extracellular environment. The TEVs consist of various vesicles, especially exosomes, apoptotic bodies, and microvesicles. These vesicles have unique cargo profiles that reflect the characteristics of the original tumor cells, including proteins, lipids, and nucleic acids, and are known to affect the functions or phenotypes of recipient cells. Previous studies have known that the TEVs carry tumor antigens, including tumor-associated antigens (TAAs) and tumor-specific antigens (TSAs), which are considered the most important components of antitumor vaccines (T. Huang, C.-X. Deng, Int. J. Biol. Sci. 2019, 15, 1-11.).

Verteporfin is a drug with the trade name Visudyne, approved with the Food and Drug Administration (FDA), which is most commonly used as a photosensitizer for photodynamic therapy, but is known to have different mechanisms in various diseases, representatively, a verteporfin-mediated YAP inhibition mechanism. Verteporfin-mediated YAP-dependent cell death has been confirmed to be immunogenic, which indicates the possibility of YAP inhibition mediating an immune response by using immunogenic molecules through immunogenic cell death (ICD).

The aforementioned background art is included or obtained by the present inventors in the process of deriving the disclosure of the present disclosure, and cannot necessarily be known art disclosed to the general public prior to the present application.

### SUMMARY

Embodiments provide a method for preparing a tumor-derived vesicle composition and a tumor-derived vesicle composition prepared thereby.

Embodiments also provide a vaccine composition or an immune adjuvant including the tumor-derived vesicles as an active ingredient.

However, aspects of the present disclosure are not limited to the aforementioned aspects, and other aspects which are not mentioned can be clearly understood to those skilled in the art from the following description.

According to an aspect, there is provided a tumor-derived vesicle composition prepared through the following steps:
1) providing a cancer cell line;
2) treating the cancer cell line with verteporfin (VP); and
3) extracting extracellular vesicles from the cancer cell line.

According to an aspect, the cancer cell line may be at least any one selected from the group consisting of E.G7-OVA, MOC2, and 4T1.

According to an aspect, the verteporfin may be treated at 10 to 200 ng/ml.

According to one aspect, the extracellular vesicles may have a diameter of 50 to 200 nm.

According to an aspect, the extracellular vesicles may be attenuated yet immunogenically potentiated.

According to one aspect, the extracellular vesicles may overexpress damage-associated molecular patterns (DAMPs), and the DAMP may be at least any one selected from 70 kilodalton heat shock proteins (Hsp70) and high mobility group box 1 (HMGB1).

According to an aspect, the extracellular vesicles may activate dendritic cells.

According to another aspect, there is provided an anticancer vaccine composition including the extracellular vesicles as an active ingredient.

According to one aspect, the anticancer vaccine composition may induce tumor-specific immunity.

According to yet another aspect, there is provided an anticancer immune adjuvant composition including the extracellular vesicles as an active ingredient.

According to one aspect, the anticancer immune adjuvant composition may induce long-term memory to prevent cancer recurrence.

According to yet another aspect, there is provided a method for preparing a tumor-derived vesicle composition including the following steps:
1) providing a cancer cell line;
2) treating the cancer cell line with verteporfin (VP); and
3) extracting extracellular vesicles from the cancer cell line.

According to an aspect, the cancer cell line may be at least any one selected from the group consisting of E.G7-OVA, MOC2, and 4T1.

According to an aspect, the verteporfin may be treated at 10 to 200 ng/ml.

According to still another aspect, there is provided a method for preparing a subject-customized vaccine composition including the following steps:
1) collecting tumor cells from a subject;
2) treating the tumor cells with verteporfin; and
3) extracting extracellular vesicles from the tumor cells.

According to embodiments, the present disclosure relates to a method for preparing a tumor-derived vesicle composition and a tumor-derived vesicle composition prepared thereby. Due to the treatment of verteporfin, which is a feature of the preparation method of the present disclosure, TEVs secreted from tumor cells are attenuated yet immunogenically potentiated, and thus can be used as an anticancer vaccine or an immune adjuvant, and can also be used for preparing a subject-customized vaccine.

The effects of the present disclosure are not limited to the aforementioned effects, and other effects, which are not mentioned above, will be clearly appreciated by a person having ordinary skill in the art from the following description.

### BRIEF DESCRIPTION OF THE DRAWINGS

These and/or other aspects, features, and advantages of the present disclosure will become apparent and more readily appreciated from the following description of embodiments, taken in conjunction with the accompanying drawings of which:

FIG. 1 is a schematic diagram of a method for preparing attenuated yet immunogenically potentiated tumor-derived vesicles (AI-TEVs) of the present disclosure;

FIG. 2 shows cell viability of E.G7-OVA cells cultured with various concentrations of verteporfin for 24 hours;

FIG. 3 shows cell viability of MOC2 and 4T1 cells cultured with various concentrations of verteporfin for 24 hours;

FIG. 4 shows results of flow cytometry for E.G7-OVA cells cultured with verteporfin for 24 hours;

FIG. 5 shows results of Western blotting of HMGB1 and HSP70 released from a conditioned medium of E.G7-OVA cells 24 hours after verteporfin treatment;

FIG. 6 shows results of flow cytometry for calreticulin expression on the cell surface;

FIG. 7 shows results of Western blotting of HMGB1 and HSP70 released from a controlled medium 24 hours after verteporfin treatment;

FIG. 8 shows the size distribution and particle number of TEVs evaluated using nanoparticle tracking assay;

FIG. 9 shows cryo-TEM (cryogenic transmission electron microscopy) images of C-TEV and AI-TEV (scale bar: 200 nm);

FIG. 10 shows the size and particle number of TEVs, as characteristic analysis of C-TEV and AI-TEV in MOC2 and 4T1 cancer cells;

FIG. 11 shows cryo-TEM images of C-TEV and AI-TEV secreted from 4T1 cells;

FIG. 12 shows results of Western blotting of cell lysates and TEVs for detecting EV markers Tsg101, CD81, and Alix along with a negative marker calnexin;

FIG. 13 shows results of Western blotting of HMGB1 and HSP70 released from a controlled medium 24 hours after verteporfin treatment;

FIG. 14 is a scatter plot for comparing proteomic cargos of C-TEV and AI-TEV;

FIG. 15 shows results of Western blotting of E.G7-OVA and MOC2 cells treated with verteporfin for 24 hours;

FIG. 16 is a heat map showing relative abundance proteins of E.G7-OVA TEV, especially proteins known to induce cell migration and cell proliferation directly downstream of a YAP/TAZ Hippo signaling pathway;

FIG. 17 shows results of cell proliferation analysis of E.G7-OVA and MOC2 cells treated with various concentrations of TEV for 24 hours, respectively;

FIG. 18 shows a representative microscopic image of MOC2 cells migrating through a transwell in a transwell migration assay of MOC2 cells;

FIG. 19 shows the number of migrated MOC2 cells calculated using ImageJ software in the transwell migration assay of MOC2 and E.G7-OVA cells;

FIGS. 20A-C show results showing that verteporfin inhibits the pro-tumorigenic properties of YAP and 4T1 TEV in 4T1 cells, which shows FIG. 20A) Western blotting after 24-hour treatment of 4T1 cells with verteporfin, FIG. 20B) relative abundance proteins of 4T1 TEV, and FIG. 20C) cell proliferation analysis of 4T1 cells treated with different concentrations of TEV for 24 hours;

FIGS. 21A-B show FIG. 21A) representative microscopic images (crystal violet staining. Magnification, X10) of 4T1 cells that migrated through a transwell, and FIG. 21B) the number of migrated 4T1 cells calculated using ImageJ software;

FIGS. 22A-B show FIG. 22A) a therapeutic vaccination scheme of TEV, and FIG. 22A B) an average tumor growth curve and individual tumor growth curves for each group;

FIGS. 23A-B show that AI-TEV is rich in antigens and adjuvants to have enhanced ability to prime dendritic cells, which show FIG. 23A) Western blot analysis of TEV detecting ovalbumin (OVA) and DAMPs (HMGB1, HSP70), FIG. 23B) a heatmap showing relative abundance of various other DAMPs;

FIGS. 24A-E show FIG. 24A) quantification of dsDNA in E.G7-OVA TEV using a QuantiFluor dsDNA system, and FIG. 24B) to FIG. 24E) results of flow cytometry or ELISA experiments;

FIGS. 25A-C show verifying that MOC2 TEV has enhanced DC priming ability, which show FIG. 25A) dsDNA quantification using a QuantiFluor dsDNA system, FIG. 25B) flow cytometry for pIRF3 and pTBK1 expression in CD11c+ BMDCs treated with MOC2 TEV for 48 hours, and FIG. 25C) flow cytometry for DC maturation markers (CD40, CD86) in CD11c+ BMDCs treated with E.G7-OVA TEV for 48 hours;

FIGS. 26A-D show that AI-TEV serves as an effective preventive vaccine to induce tumor-specific immunity, which show FIG. 26A) vaccination schedule of E.G7-OVA derived TEV, FIG. 26B) average tumor growth curves (n=7 to 9 per group) and individual tumor growth curves for each group, FIG. 26C) vaccination experimental design scheme for E.G7-OVA-derived TEV-induced immunity analysis, and FIG. 26D) results of splenocytes stimulated with UV-irradiated cancer cells for 5 hours and analyzed by flow cytometry;

FIGS. 27A-E show that AI-TEV serves as a preventive vaccine against MOC2 to induce tumor-specific immunity, which show FIG. 27A) TEV vaccination scheme, FIG. 27B) average tumor growth curves (n=4 to 6 per group) and individual tumor growth curves, FIG. 27C) vaccination experimental design scheme for MOC2-derived TEV-induced immunity analysis, FIG. 27D) results splenocytes stimulated with UV-irradiated cancer cells for 5 hours and analyzed by flow cytometry, and FIG. 27E) results of flow cytometry for the percentage of CD86+ cells among CD11c+ cells and the percentage of CD44+ cells among CD3+CD8+ cells in TDLN;

FIG. 28 shows a flow cytometry gating strategy for analysis of tumor cell-pulsed splenocytes;

FIGS. 29A-D show FIG. 29A) results of flow cytometry for splenocytes stained with carboxyfluorescein succinimidyl ester (CFSE) and stimulated with UV-irradiated cancer cells for 72 hours, FIG. 29B) results of flow cytometry for the percentage of CD40+ and CD86+ cells among CD11c+ cells in TDLN, FIG. 29C) results of flow cytometry for the percentage of CD44+ cells among CD3+CD8+ cells in TDLN, and FIG. 29D) results of flow cytometry for OVA-tetramer-binding CD8+ cells in TDLN;

FIG. 30 shows a flow cytometry gating strategy for TDLN analysis;

FIGS. 31A-E show FIG. 31A) an incomplete resection model of E.G7-OVA tumors to observe an effect of vaccination on tumor recurrence, FIG. 31B) photographs of incomplete resections of tumors of mice, FIG. 31C) average tumor growth curves (n=5 to 6 per group) and individual tumor growth curves, FIG. 31D) flow cytometry to evaluate the percentages of CD44+CD8+ T cells, CD44hiCD62Lhi T cells, and CD44hiCD62Llo T cells, and FIG. 31E) flow cytometry results of OVA-tetramer-binding CD8+ cells in TDLN;

FIG. 32 shows a flow cytometry gating strategy for analysis of memory T cells in the spleen;

FIGS. 33A-F show FIG. 33A) a customized cancer vaccine model scheme, FIG. 33B) Western blotting of cell lysates and TEVs to detect EV markers Tsg101, CD81 and Alix along with a negative marker calnexin, FIG. 33C) Western blotting of TEVs to detect OVA protein and DAMPs (HMGB1, HSP70), FIG. 33D) quantification of dsDNA loaded into TEVs using a QuantiFluor dsDNA system, FIG. 33E) vaccination scheme of mice inoculated with E.G7-OVA cells, FIG. 33F) average tumor growth curves of tumor size (n=6 to 7 per group) and individual tumor growth curves for each group; and

FIGS. 34A-C show FIG. 34A) results of flow cytometry for the spleen of vaccinated mice stimulated with UV-irradiated cancer cells for 5 hours, FIG. 34B) results of flow cytometry for the percentage of CD40+ and CD86+ cells among CD11c+ cells in TDLN harvested from vaccinated mice, and FIG. 34C) results of flow cytometry for OVA-tetramer-binding CD8+ cells from TDLN.

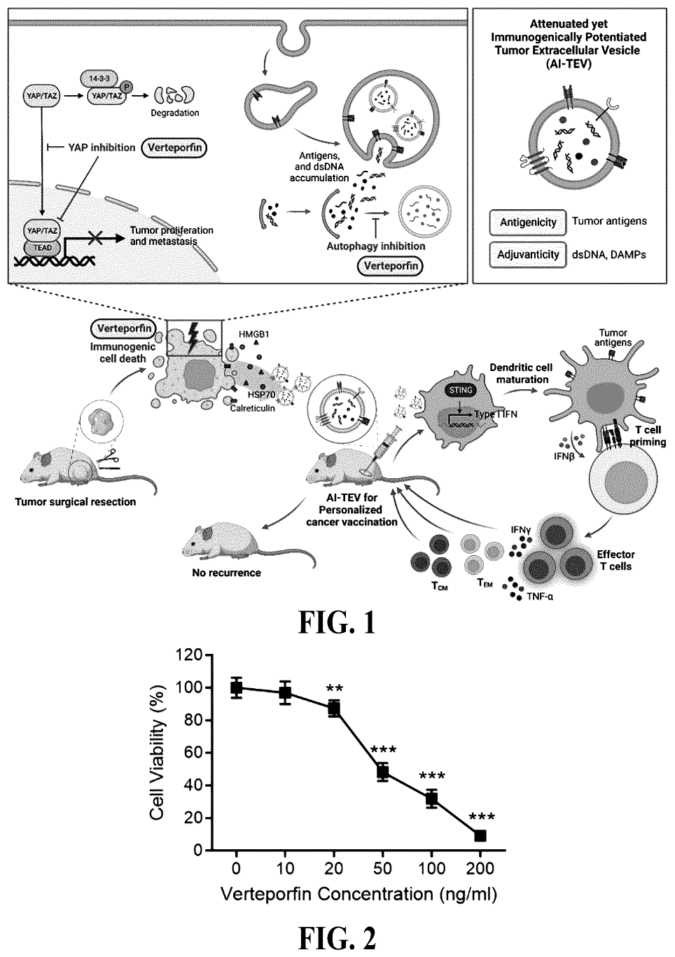

### DETAILED DESCRIPTION

The present inventors expected that verteporfin inhibited both autophagy and YAP protein in tumor cells and simultaneously induced ICD to nullify the original pro-tumorigenic properties while causing tumor cells to secrete TEVs containing a large amount of dsDNA and other immunogenic molecules such as tumor antigens and danger-associated molecular patterns (DAMPs), and intended to complete and provide attenuated yet immunogenically potentiated TEVs (AI-TEVs) as the present disclosure.

According to an aspect, the present inventors provide a tumor-derived vesicle composition prepared through the following steps:
1) providing a cancer cell line;
2) treating the cancer cell line with verteporfin (VP); and
3) extracting extracellular vesicles from the cancer cell line.

The term “extracellular vesicles” as used herein refer to a heterogeneous group of membrane structures derived from the outward budding of the plasma membrane or endosomal system of a cell. The extracellular vesicles may include small extracellular vesicles, exosomes, and microvesicles. Extracellular vesicles derived from tumor cells are referred to herein as “tumor-derived vesicles”, which may be used interchangeably with “tumor-derived extracellular vesicles”, “TEVs”, “tumor-derived exosomes”, and the like. These extracellular vesicles are typically secreted from tumor cells into their surrounding microenvironments. The extracellular vesicles include small extracellular vesicles (50 to 200 nm), microvesicles (0.2 to 1 μm), exomers and exosomes (30 to 150 nm), and oncosomes (1 μm up to 10 μm). In one example, the tumor-derived extracellular vesicles are tumor-derived small extracellular vesicles (50 to 200 nm), microvesicles (0.2 to 1 μm), or exosomes (30 to 150 nm). In another example, the tumor-derived extracellular vesicles are tumor-derived exosomes. In another example, the tumor-derived extracellular vesicles are exomers.

The “extracting of the extracellular vesicles” may be performed by methods known in the art, desirably by centrifugation, or optionally by using a commercially available exosome elution/precipitation solution such as Exoquick.

According to an aspect, the cancer cell line may be desirably a cancer cell line in which verteporfin exhibits cell death efficacy, and most desirably, at least any one selected from the group consisting of E.G7-OVA, MOC2, and 4T1, but is not limited thereto.

According to an aspect, the verteporfin may be treated at 10 to 200 ng/mL, desirably at 20 to 200 ng/ml, and most desirably, at 20 ng/ml in the case of E.G7-OVA cells, and at 200 ng/ml in the case of MOC2 and 4T1 cells. As disclosed in Example 2 and the like of the present disclosure, treatment with an appropriate amount of verteporfin is an important element of the preparation method that attenuates tumor cells while preventing the tumor cells from being completely killed and imparts potentiated immunogenicity.

According to one aspect, the extracellular vesicles may have a diameter of 50 to 200 nm, but are not limited thereto.

According to an aspect, the extracellular vesicles may be attenuated yet immunogenically potentiated. The term “attenuated” used herein means that the biotoxicity of a pathogen has been artificially weakened, and means that the pathogen does not cause disease in the body by mutating genes involved in essential metabolism of the pathogen, and the like, and only the immune system is stimulated to induce immunogenicity. For the purpose of the present disclosure, the attenuation means that the tumorigenicity has been weakened, as disclosed in Example 3 below, etc. The term “immunogenically potentiated” used herein means more actively inducing an immune response in the body, and for the purpose of the present disclosure, the potentiation of immunogenicity may be activating dendritic cells, enhancing cross-presentation, and inducing a sustained immune response, as disclosed in Example 4 below, etc.

According to one aspect, the extracellular vesicles may overexpress damage-associated molecular patterns (DAMPs), and the DAMP may be at least any one selected from 70 kilodalton heat shock proteins (Hsp70) and high mobility group box 1 (HMGB1). As verified in Example 2 below, TEV containing a large amount of these DAMP biomolecules exhibits strong immunogenicity and may be involved in immunogenic cell death (ICD).

According to an aspect, the extracellular vesicles may activate dendritic cells.

According to yet another aspect, there is provided an anticancer vaccine composition including the extracellular vesicles as an active ingredient. The anticancer vaccine composition may be a preventive vaccine or a therapeutic vaccine. The preventive vaccine may be administered to a subject who is not in a pathological condition related to a tumor to prevent or suppress the occurrence, growth, metastasis, and recurrence of the tumor. The therapeutic vaccine may be a vaccine that may effectively suppress the growth, metastasis, and recurrence of the tumor for a subject who is already in a pathological condition related to the tumor, and may be used to treat and prevent diseases related to pathological conditions, etc. or symptoms related with the conditions, induce a protective immune response thereto, or improve the same. As verified in Examples 5 and 6 below, etc., the composition including the TEVs of the present disclosure has an effect that can be applied as a preventive or therapeutic vaccine.

According to one aspect, the anticancer vaccine composition may induce tumor-specific immunity. The induction of tumor-specific immunity is as disclosed in Example 5 below, and may be confirmed through a ratio of activated effector CD8+ T cells.

According to yet another aspect, there is provided an anticancer immune adjuvant composition including the extracellular vesicles as an active ingredient. The term “immunomodulator”, or adjuvant used herein refers to a substance that enhances an immune response caused by an antigen. The adjuvant may enable a small amount of antigen in a vaccine to exhibit the same effect, or induce a favorable immune response through a mechanism known or unknown in the art. In particular, the immunomodulator used herein may be desirably used as an auxiliary immunotherapy that increases tumor-specific long-term memory to prevent cancer recurrence, and may be administered in combination with commercialized chemotherapy or immunotherapy.

According to yet another aspect, there is provided a method for preparing a tumor-derived vesicle composition including the following steps:
1) providing a cancer cell line;
2) treating the cancer cell line with verteporfin (VP); and
3) extracting extracellular vesicles from the cancer cell line.

According to an aspect, the cancer cell line may be at least any one selected from the group consisting of E.G7-OVA, MOC2, and 4T1, and the specific meaning is as described above.

According to an aspect, the verteporfin may be treated at 10 to 200 ng/mL, and the specific meaning of the numerical value is as described above.

According to still another aspect, there is provided a method for preparing a subject-customized vaccine composition including the following steps:
1) collecting tumor cells from a subject;
2) treating the tumor cells with verteporfin; and
3) extracting extracellular vesicles from the tumor cells.

In the present disclosure, the term “subject” refers to desirably a living organism that is suffering from, has been treated for, or is susceptible to a disease, such as a solid cancer, which may be prevented or treated by the administration of the vaccine composition of the present disclosure, and includes both humans and animals. The subject includes mammals (e.g., mice, monkeys, horses, cows, pigs, dogs, cats, etc.), but is not limited thereto and desirably humans.

As used herein, the term “prevention” means all actions that inhibit or delay the occurrence, spread or recurrence of cancer by administering the composition of the present disclosure, and the “treatment” means all actions that improve or beneficially change the symptoms of the disease by administering the composition of the present disclosure.

As used herein, the term “pharmaceutical composition” means a composition prepared for preventing or treating the diseases, and may be formulated and used in various forms according to conventional methods. For example, the pharmaceutical composition may be prepared for oral formulations, such as powders, granules, tablets, capsules, suspensions, emulsions, syrups, and the like, and formulated and used in the form of external preparations, suppositories, and sterile injection solutions.

In the present disclosure, “including as the active ingredient” means that the corresponding ingredient is included in an amount required or sufficient to realize a desired biological effect. In actual application, the amount included as the active ingredient may be determined by considering matters that do not cause other toxicities, as an amount for treating a target disease, and for example, may vary depending on various factors, such as a disease or condition to be treated, a type of composition to be administered, the size of a subject, and the severity of the disease or condition. Effective amounts of individual compositions may be determined empirically without undue experimentation by those skilled in the art to which the present disclosure pertains.

In addition, the pharmaceutical composition of the present disclosure may further include one or more pharmaceutically acceptable carriers in addition to the above-described active ingredients according to each formulation.

The pharmaceutically acceptable carrier may be saline, sterile water, a Ringer's solution, buffered saline, a dextrose solution, a maltodextrin solution, glycerol, ethanol, and mixtures of at least one ingredient thereof, and if necessary, may also further include other conventional additives such as antioxidants, buffers, bacteriostats, etc. In addition, the pharmaceutical composition may also be prepared in injectable formulations such as aqueous solutions, suspensions, and emulsions, pills, capsules, granules, or tablets by further adding a diluent, a dispersant, a surfactant, a binder, and a lubricant. Furthermore, the pharmaceutical composition may also be desirably prepared according to each disease or ingredient by a suitable method of the art, or using a method disclosed in Remington's Pharmaceutical Science (Mack Publishing Company, Easton PA).

The composition of the present disclosure may be administered orally or parenterally in a pharmaceutically effective amount according to a desired method. As used herein, the term “pharmaceutically effective amount” refers to an amount enough to treat the disease at a reasonable benefit/risk ratio applicable to medical treatment and not to cause side effects. The effective amount level may be determined according to factors including the health condition of a patient, the severity, the activity of a drug, the sensitivity to a drug, an administration method, a time of administration, a route of administration, an emission rate, duration of treatment, and simultaneously used drugs, and other factors well-known in the medical field.

In addition, the present disclosure may provide a method for preventing or treating cancer including administering a tumor-derived vesicle composition to a subject.

As used herein, the term “subject” is any mammal, such as livestock or humans required for prevention, treatment, and/or diagnosis of the diseases without limitation, and may be desirably humans, and may be used interchangeably with the meaning of the “subject” described in the above paragraph.

As used herein, the term “administration” means providing a predetermined material to a patient by any appropriate method. The pharmaceutical composition of the present disclosure may be formulated in various forms for administration to a subject, and a representative formulation for parenteral administration is an injectable formulation, desirably an isotonic aqueous solution or suspension. The injectable formulation may be prepared according to techniques known in the art by using suitable dispersing or wetting agents and suspending agents. For example, the injectable formulation may be formulated for injection by dissolving each ingredient in saline or buffer. In addition, the formulations for oral administration include, for example, ingestible tablets, buccal tablets, troches, capsules, elixirs, suspensions, syrups, wafers, and the like. In addition to the active ingredients, these formulations may include diluents (e.g., lactose, dextrose, sucrose, mannitol, sorbitol, cellulose and/or glycine) and lubricants (e.g., silica, talc, stearic acid and magnesium or calcium salts thereof and/or polyethylene glycol). The tablets may include a binder such as magnesium aluminum silicate, starch paste, gelatin, tragacanth, methylcellulose, sodium carboxymethylcellulose and/or polyvinylpyrrolidine. In some cases, the tablets may further include a disintegrant such as starch, agar, alginic acid or sodium salt thereof, an absorbent, a coloring agent, a flavoring agent and/or a sweetening agent. The formulations may be prepared by conventional mixing, granulating or coating methods.

In addition, the pharmaceutical composition or the vaccine composition of the present disclosure may further include adjuvants such as preservatives, hydrating agents, emulsifying accelerators, and salts or buffers for osmotic pressure control, and other therapeutically useful substances, and may be formulated according to conventional methods.

The pharmaceutical composition or the vaccine composition according to the present disclosure may be administered through various routes including oral, transdermal, subcutaneous, intravenous or intramuscular route, and the dose of the active ingredient may be appropriately selected according to various factors such as a route of administration, the age, sex, and weight of a patient, and the severity of a patient. In addition, the composition of the present disclosure may be administered in combination with known compounds capable of increasing the desired effect.

The total effective dose of the pharmaceutical composition or the vaccine composition of the present disclosure may be administered to a patient in a single dose, or may be administered according to a fractionated treatment protocol in which a multiple dose is administered for a long period of time. In the pharmaceutical composition or the vaccine composition of the present disclosure, the content of the active ingredient may vary depending on the severity of disease, but generally, the pharmaceutical composition or the vaccine composition may be repeatedly administered several times a day at an effective dose of 100 μg to 3,000 mg when administered once based on adults, but is not limited to the corresponding content. In the concentration of the pharmaceutical composition or the vaccine composition, an effective dose to the subject may be determined by considering various factors such as the age, weight, health condition, and sex of a patient, the severity of a disease, diet and excretion rate as well as the route of administration of a drug and the number of treatments.

In addition, the pharmaceutical composition or the vaccine composition according to the present disclosure is not particularly limited to the formulation, the administration route, and the administration method thereof, as long as the effects of the present disclosure are exhibited.

The terms used in the embodiments are used for the purpose of description only, and should not be construed to be limited. A singular expression includes a plural expression unless otherwise defined differently in a context. In the present specification, it should be understood that term “including” or “having” indicates that a feature, a number, a step, an operation, a component, a part or the combination thereof described in the specification is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art to which embodiments pertain. Terms which are defined in a generally used dictionary should be interpreted to have the same meaning as the meaning in the context of the related art, and are not interpreted as ideal or excessively formal meanings unless otherwise defined in the present application.

The present disclosure may have various modifications and various embodiments, and specific embodiments will be hereinafter illustrated in the drawings and described in detail in the detailed description. However, the present disclosure is not limited to specific embodiments, and it should be understood that the present disclosure covers all the modifications, equivalents and replacements within the idea and technical scope of the present disclosure. In describing the present disclosure, when it is determined that a detailed description of related known arts may obscure the gist of the present disclosure, the detailed description will be omitted.

### Example 1. Experimental Method

### Example 1-1. Cell Culture

E.G7-OVA mouse thymoma cells and 4T1 mouse breast cancer cells were purchased from ATCC (Manassas, VA, USA) and cultured in Roswell park memorial institute medium (RPMI 1640; SH30255.01; Hyclone, Logan, UT, USA) supplemented with the following: 10% fetal bovine serum (FBS; 12483-020; Gibco, Billings, Montana, USA) and 1% antibiotic-antimycotic (15240-062; Gibco).

MOC2 cells were also purchased from ATCC and cultured in Iscove's modified Dulbecco's medium (SH3022902, Hyclone) supplemented with the following: 5% FBS, 1% penicillin-streptomycin solution (SV30010; Hyclone), 31.3% Ham's nutrient mixture F12 (SH30026.01; Hyclone), 5 μg/mL insulin (16634-50 mg; Sigma-Aldrich, Burlington, MA, USA), 0.04 μg/mL hydrocortisone (H0135-1 mg; Sigma-Aldrich), and 0.005 μg/mL recombinant human epidermal growth factor (EGF; C029; Novoprotein, Suzhou, China). All of the cells were cultured at 37° C. and 5% CO₂.

### Example 1-2. TEV Preparation and Isolation

When cancer cells reached 80 to 90% confluence, the cancer cells were cultured for 24 hours in a serum-free medium containing verteporfin (20 ng/ml for E.G7-OVA cells and 200 ng/mL for 4T1 and MOC2 cells). The verteporfin was purchased from Sigma-Aldrich The cell supernatant was subjected to a series of centrifugation steps. (SML0534). Microvesicles and cell debris were removed at 300×g for 10 minutes, 2000×g for 10 minutes, and 10,000×g for 30 minutes. Subsequently, the resulting supernatant was filtered through a 0.22-μm pore filter (431118; Corning®, Corning, NY, USA), dialyzed against PBS through tangential flow filtration (KrosFlo® KR2i, Repligen, Boston, MA, USA) using a MIDIKROS filter (41.5 cm 300 K MPES 0.5 mm; Repligen), and centrifuged at 150,000×g for 2 hours. The pellet was reduced in PBS containing a protease inhibitor cocktail (PIC, 11697498001, Roche, Basel, Switzerland) and stored at 4° C.

### Example 1-3. Nanoparticle Tracking Analysis

The size and number of TEVs were measured using Zeta View® (Particle Metrix, Meerbusch, Germany) and the corresponding software (Zeta View 8.02.28). After calibrating the Zeta View system with polystyrene beads (3090A, ThermoFisher Scientific, Waltham, MA, USA), exosomes were diluted and loaded to obtain size distribution and particle counts. Samples were analyzed at 11 different locations throughout the chamber.

### Example 1-4. Cryo-Transmission Electron Microscopy

TEVs were visualized using cryo-transmission electron microscopy (Tecnai Systems, Philips, Holland). Purified TEVs were deposited on a thin carbon film of a copper grid and then cryogenically preserved using a Vitrobot (FP5350, FEI).

### Example 1-5. Immunoblotting

Protein concentrations of the samples were quantified using a DCTM protein assay kit (5000111, Bio-Rad, Hercules, CA, USA). The samples were dissolved using radioimmunoprecipitation buffer (9806S; Cell Signaling Technology, Danvers, MA, USA) and subjected to SDS-PAGE. The samples were loaded onto a sodium dodecyl sulfate polyacrylamide gel, separated by gel electrophoresis, and transferred to a nitrocellulose membrane. The membrane was blocked with 5% skim milk and incubated overnight at 4° C. with primary antibodies against the following: Tsg101 (sc-22774; Santa Cruz, Dallas, TX, USA), YAP (sc99010), CD81 (sc-166029), ALIX (sc-53540), calnexin (ab22595; Abcam, Cambridge, United Kingdom), OVA (0220-1682; Bio-Rad), HMGB1 (ab18256; Abcam), HSP70 (ab181606; Abcam), or B-actin (4967S; Cell Signaling Technology). After washing five times (5 min each) with Tris-buffered saline containing 0.05% Tween-20 (TBS/T), the membrane was incubated with anti-mouse peroxidase (1:3000; A4416; Sigma-Aldrich) or anti-rabbit peroxidase (1:3000; A0545) secondary antibody at 20 to 25° C. for 1 hour. The membrane was then incubated with an ECL substrate (1705061, Bio-Rad) and visualized using a ChemiDoc™ Touch Imaging System (Bio-Rad).

### Example 1-6. Cell Viability Assay

Cells were seeded in a 96-well plate (30096; SPL Life Sciences, Pochon, South Korea) (5,000 cells/well) and treated with various doses of verteporfin in a serum-free medium for 24 hours. Cell viability was measured using Cell counting kit-8 (CCK-8) assay (CK04; Dojindo, Kumamoto, Japan), and the absorbance was measured at 450 nm using a microplate reader (SpectraMAX 340; Molecular Devices, San Jose, CA, USA).

### Example 1-7. Calreticulin Assay

Cells were seeded in a 6-well plate (30006; SPL Life Sciences, 5×10⁵ cells/well) and treated with verteporfin in a serum-free medium for 3 hours. The cells were fixed with 0.25% paraformaldehyde for 5 minutes at 4° C. and then blocked with 3% bovine serum albumin for 15 minutes at 4° C. A calreticulin antibody (ab2907; Abcam) was added to the samples and incubated at 4° C. for 45 minutes. After washing with PBS, the samples were added with Alexa Fluor 488-conjugated secondary antibody (711-545-152; Jackson ImmunoResearch, West Grove, PA, USA) and incubated at 4° C. for 30 minutes. The samples were washed with Dulbecco's phosphate-buffered saline (DPBS; LB001-02; Welgene, Taipei, China) and stained with propidium iodide (PI; P4864; Sigma-Aldrich) at 44° C. for 15 minutes to determine cell viability. Calreticulin expression on the cell membrane was evaluated using flow cytometry gating of PI-negative cells.

### Example 1-8. Evaluation of Effect of TEV on Dendritic Cell (DC)

Bone marrow cells were extracted from 6 to 8-week-old C57BL/6 mice (Orient Bio, Gwangju, South Korea) and cultured in RPMI 1640 medium (LM011-01; Welgene) supplemented with 10% FBS (12483-020; Gibco) and 1% antibiotic-antimycotic solution (15240-062, Gibco). The cells were differentiated using 20 ng/ml recombinant mouse granulocyte-macrophage colony-stimulating factor (GM-CSF; 315-03; PeproTech, Cranbury, NJ, USA), 20 ng/mL recombinant mouse IL-4 (214-14; PeproTech), and 0.1% β-mercaptoethanol (21985023, Gibco) for 7 days. To confirm DC maturation and activation of a cGAS-STING pathway, differentiated DCs were co-cultured with 10 μg/mL TEV for 24 hours on day 8 and then analyzed by flow cytometry using the following: APC-conjugated anti-CD11c (117310, BioLegend, San Diego, CA, USA) and mature marker PE-conjugated anti-CD40 (124610, BioLegend) and anti-CD86 (105008, BioLegend). Cross-presentation was analyzed using anti-H-2Kb antibody conjugated to SIINFEKL (141603; BioLegend). The cells were fixed and permeabilized using a CytoFix/CytoPerm kit (BD 554714; BD Biosciences, Franklin Lakes, NJ, USA), and then intracellularly stained using the following: anti-phospho-TBK1/NAK (13498; Cell Signaling Technology) and anti-phospho-IRF3 (83611S; Cell Signaling Technology) antibodies. Supernatants of the TEV-treated DCs were harvested, and the concentration of IFN-γ was detected using a mouse IFN-β Quantikine ELISA kit (MIFNB0; R&D systems, NE Minneapolis, MN, USA).

### Example 1-9. dsDNA Quantification

EVs were incubated with Triton-X and proteinase K at 50° C. for 30 minutes. Thereafter, dsDNA was quantified using the QuantiFluor® dsDNA System (E2671, Promega, Madison, WI, USA) and the GloMax® Discover microplate reader (Promega).

### Example 1-10. Transwell Assay

For migration assay, 6.5 mm Transwell® (CLS3422; Corning®) with 8.0 μm pore polyester membrane inserted into an empty 24-well plate was used. For invasion assay, the membrane was coated with 0.5 mg/mL matrigel and dried at 37° C. for 24 hours before use. Cancer cells were seeded in an apical chamber with 1% FBS, and a basal chamber was filled with a serum-free medium. The cells were co-cultured with TEV (10 μg/mL) in the apical chamber. After 24 hours, the transwell membrane was washed twice with PBS, fixed with 4% paraformaldehyde, washed again with PBS, and stained with 0.1% crystal violet. After washing with PBS, the membrane was observed under an optical microscope. The stained cells were counted using ImageJ software (National Institutes of Health, Laboratory for Optical and Computational Instrumentation).

### Example 1-11. Proteomics

The concentration of TEV was quantified using a Pierce™ BCA protein assay kit (23225, Thermo Fisher Scientific). After digestion, the samples were eluted with a solution consisting of 80% acetonitrile and 0.1% formic acid in water (Honeywell, Charlotte, NC, USA) according to the manufacturer's instructions. Subsequently, the samples were reduced to a 0.1% formic acid aqueous solution and analyzed using a Q-Exactive Orbitrap hybrid mass spectrometer (Thermo Fisher Scientific) with an Ultimate 3000 system (Thermo Fisher Scientific). The Thermo MS/MS raw data file for each analysis was searched using Proteome Discoverer™ software (version 2.5) and a Musculus database was provided by UniProt (https://www.uniprot.org/). The relevant proteomic data ratios were evaluated using ExDEGA version 3.0.1 (ebiogen, Seoul, Korea).

### Example 1-12. Animal Experiment

Male C57BL/6 (6 to 7-week-old) and female BALB/c (6 to 7-week-old) mice were purchased from Orient Bio and bred under environmentally controlled conditions (23±2° C., 55±10% relative humidity, 12-h light/dark cycle) capable of freely taking food and water in a specific pathogen-free animal facility at the Korea Institute of Science and Technology (KIST, Seoul, Republic of Korea). The animal experiment protocol was approved by the KIST Association for Assessment and Accreditation of Laboratory Animal Care (Approval No. KIST-IACUC-2020-095).

### Example 1-13. In Vivo Tumor Model

A therapeutic vaccination model was established by subcutaneously inoculating 5×10⁵ E.G7-OVA tumor cells into the left flanks of C57BL/6 mice. Five days after inoculation, 20 μg of TEV was injected subcutaneously near the tumor inoculation site once every two days for a total of five times. A preventive vaccination model was established by subcutaneously injecting 40 μg of TEV into the right flanks of C57BL/6 or BALB/c mice once a week for a total of two times. One week after the last vaccination, approximately 5×10⁵ tumor cells were subcutaneously inoculated into the left flanks of mice and observed every two days. The tumor size was determined by calculating the area using Equation of (width)²×(length)/2, and mice with tumors>2,000 mm³ were euthanized and TDLN and spleen were harvested for analysis.

A tumor recurrence model was established by subcutaneously inoculating approximately 5×10⁵ E.G7-OVA tumor cells into the left flanks of C57BL/6 mice. After 7 days, when the tumor size grew to approximately 100 mm³, approximately 10% of the tumor mass remained in the tumor bed by incompletely resecting the tumor. Mice with tumors that did not reach the appropriate size were excluded. Six days after inoculation, 20 μg of TEV was injected subcutaneously near the tumor inoculation site once every two days for a total of five times. The mice were euthanized, and TDLN and spleen were harvested for analysis 16 days after resection.

A customized cancer vaccine model was established by subcutaneously inoculating approximately 1×10⁶ E.G7-OVA tumor cells into the right flanks of C57BL/6 mice. After 7 days, when the tumor size grew to approximately 300 mm³, the tumor was completely resected and isolated using a tumor isolation kit (130-096-730; Miltenyi Biotec, Bergisch-Gladbach, Germany) and a GentleMACS separator (Miltenyi Biotec). Dead cells were removed using a dead cell removal kit (130-090-101; Miltenyi Biotec), and the remaining live cells were suspended in serum-containing RPMI. After 24 hours, the cells attached to the culture flask were discarded, and the suspended cells were resuspended and cultured in RPMI. TEVs isolated from these cells were injected subcutaneously into the left flank of the same mice once a week for a total of two times. One week after the last vaccination, the mice were administered with the cultured cancer cells or euthanized to monitor tumor growth, and then the spleen and TDLN were harvested.

### Example 1-14. In Vitro Analysis

To determine T cell activation, spleens were isolated from vaccinated mice, separated using a GentleMACS separator (Miltenyi Biotec), and seeded in a 96-well round-bottom plate at 5×10⁵ cells/well. The cancer cells were irradiated with UV light for 30 minutes and added to the splenocytes seeded at 1×10⁵ cells/well. After 1 hour, the cells were added with protein transport inhibitors BD GolgiStop™ (containing Monensin, BD 554724, BD Biosciences) and BD GolgiPlug™ (containing Brefeldin A, BD 555029, BD Biosciences) and incubated for additional 4 hours. Thereafter, the cells were harvested, fixed, and permeabilized using the CytoFix/CytoPerm kit (BD 554714, BD Biosciences). The cells were blocked with anti-mouse CD16/CD32 (553142, BD Biosciences) and intracellularly stained with the following antibodies purchased from BioLegend: anti-CD45.2 (109837), anti-CD3 (100221), anti-CD8a (100705), anti-CD44 (103008), anti-IFN-γ (505824), and anti-TNF-α (506308). To detect T cell proliferation, splenocytes were labeled with CFSE using a CellTrace™ CFSE cell proliferation kit (C34554, Thermo Fisher Scientific). The labeled splenocytes were seeded at 5×10⁵ cells/well in a 96-well round-bottom plate. Approximately 1×10⁵ irradiated cancer cells were stimulated with IL-2 (100 ng/mL) for 72 hours. Then, the cells were harvested and stained with the following antibodies purchased from BioLegend: anti-CD45.2 (109830), anti-CD3 (100227), and anti-CD8a (100712).

### Example 1-15. Flow Cytometry

The TDLN and spleen harvested from experimental mice were gently pulverized manually or using a GentleMACS dissociator (Miltenyi Biotec). The dissociated cells were filtered through a 40-μm strainer, red blood cells were lysed using a red blood cell lysis buffer (BioLegend), and then the cells were FC blocked with anti-mouse CD16/CD32 (553142, BD Biosciences). The following antibodies were used for flow cytometry, purchased from BioLegend: anti-CD11c (117310), anti-CD40 (124610), anti-CD86 (105008), anti-CD45.2 (109830), anti-CD3 (100222), anti-CD8a (100706), anti-CD44 (103023), and anti-CD62L (104412). Tetramer/BV421-H-2 Kb OVA (SIINFEKL) (TB-5001-4) was purchased from MBL (Sunnyvale, CA, USA). Flow cytometry data were analyzed using FlowJo (v10) software (TreeStar, San Francisco, CA, USA) and CytExpert (v2.5) software (Beckman Coulter Life Sciences, Brea, CA, USA).

### Example 1-16. Statistical Analysis

All data are expressed as mean±standard deviation (SD) or standard error of the mean (SEM) for control and experimental samples. Multiple group comparisons were performed using ANOVA and Tukey's post hoc test. Statistical significance was established using 95% (p<0.05), 99% (p<0.01), 99.9% (p <0.001), and 99.99 (p <0.0001) confidence intervals. Statistical analysis was performed using GraphPad Prism 9.5.0 (GraphPad Software, San Diego, CA, USA).

### Example 2. Preparation and Characterization of AI-TEV

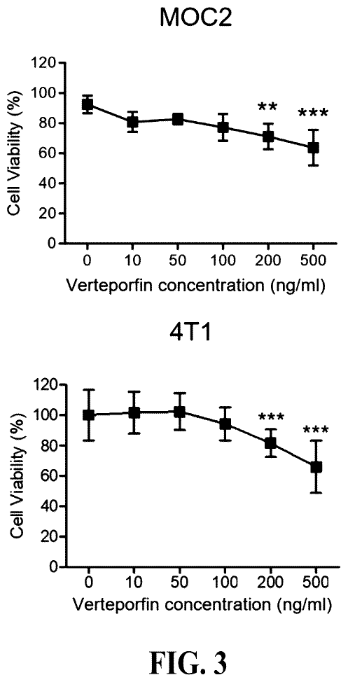

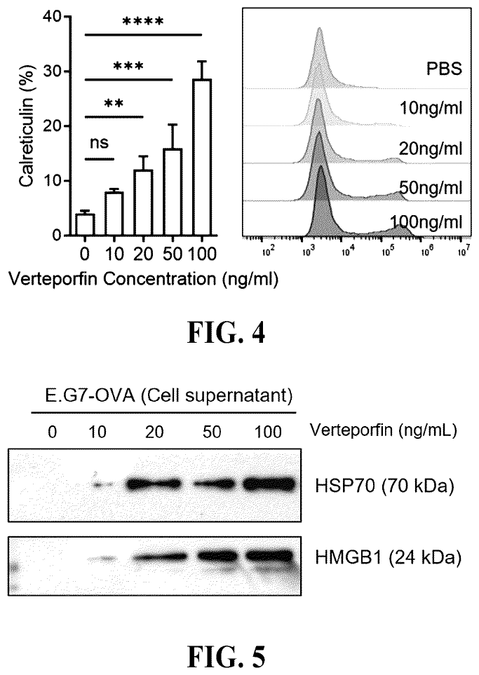

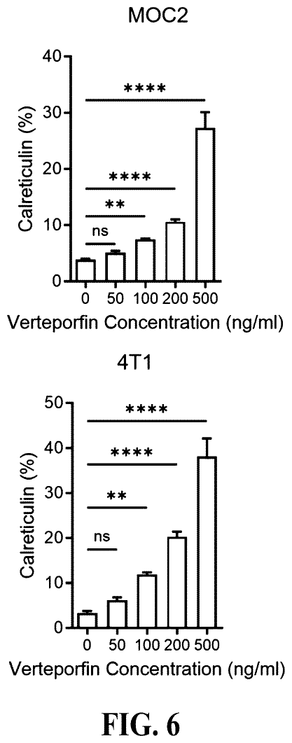

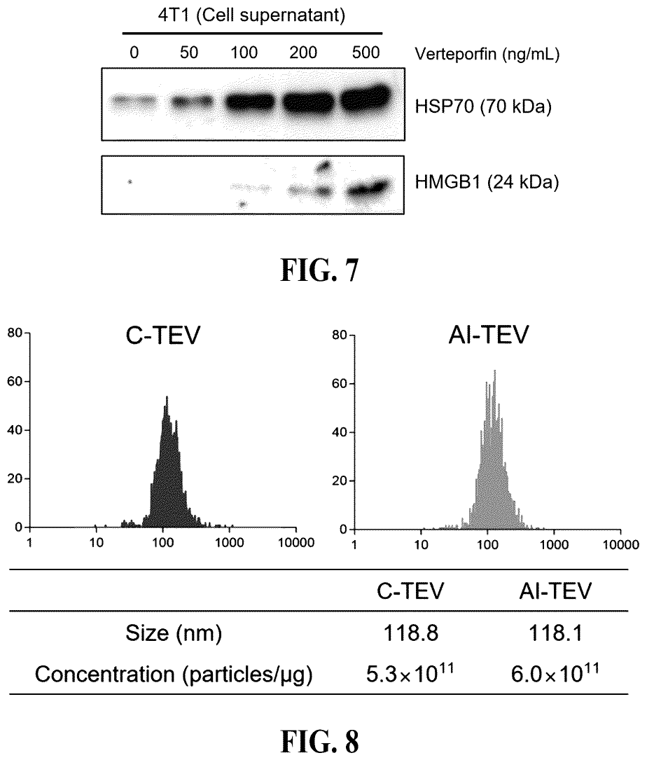

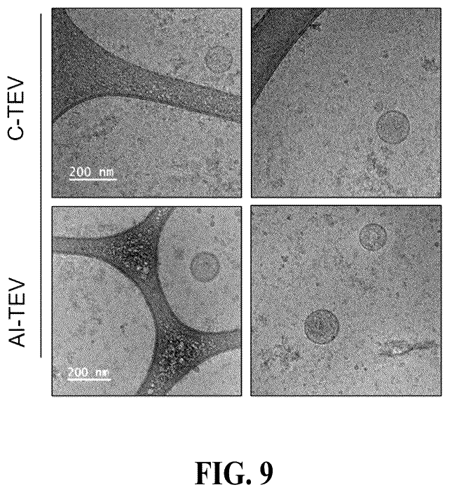

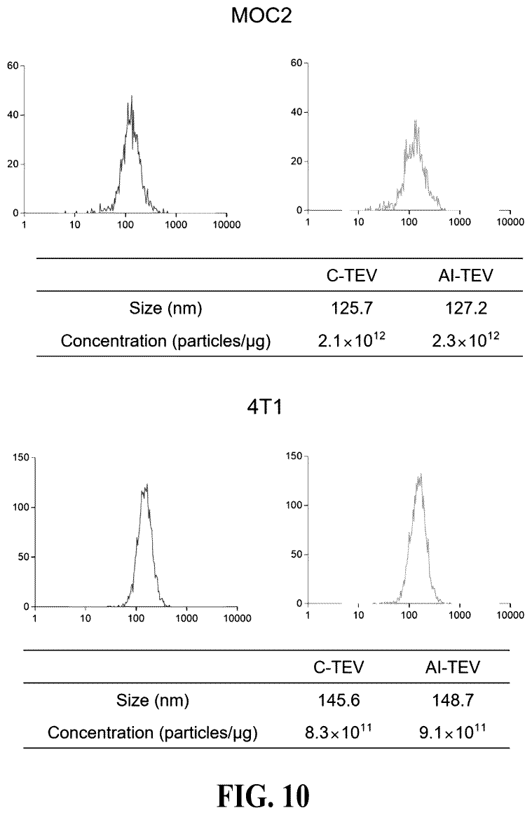

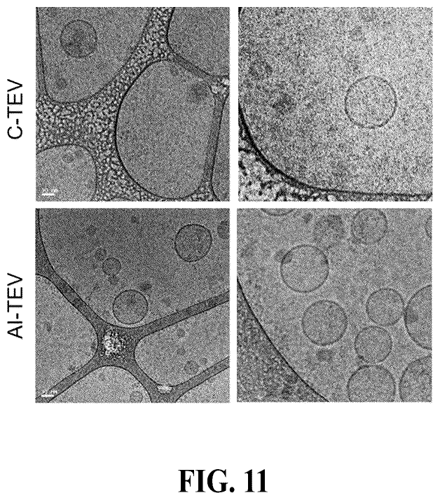

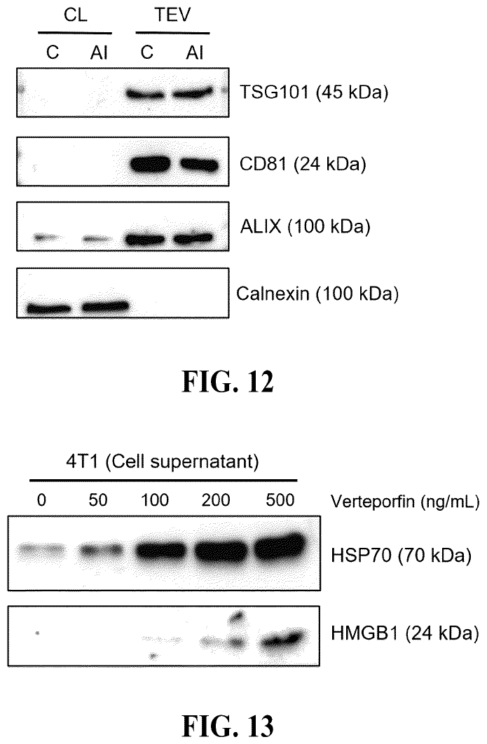

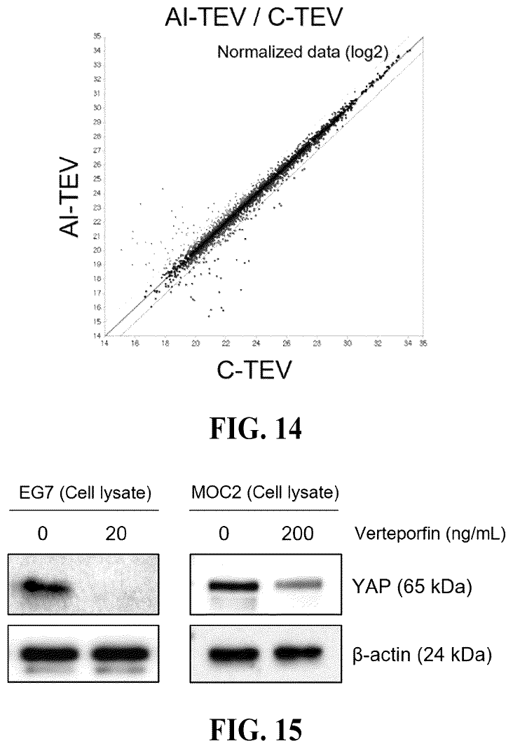

To determine appropriate conditions for AI-TEV production, three types of cancer cells were treated with various doses of verteporfin in a serum-free medium and cell viability was assessed. E.G7-OVA cells were a derivative of EL4 mouse lymphoma cells carrying a complete copy of chicken ovalbumin (OVA) mRNA. Mouse oral cancer 2 (MOC2) cells represented an aggressive form of mouse oral squamous cell carcinoma, while 4T1 cells were triple-negative, highly invasive malignant mouse breast cancer cells. The lowest dose of verteporfin that induced effective cell death was 20 ng/ml for E.G7-OVA cells and 200 ng/ml for MOC2 and 4T1 cells and cell viability was approximately 80% for all cell types (FIGS. 2 and 3). The immunogenicity of the cell death was verified based on an increase in DAMPs after verteporfin treatment. The cancer cells showed a dose-dependent increase in calreticulin expression as well as release of heat shock protein 70 (HSP70) and high mobility group box 1 (HMGB1), as widely accepted immunogenic markers defining ICD (FIGS. 4 to 7). Based on these results, to induce appropriate ICD without severely affecting cell viability, an optimal dose of verteporfin was selected in subsequent experiments (20 ng/ml for E.G7-OVA and 200 ng/ml for MOC2 and 4T1 cells). Tumor cells were cultured in a serum-free medium for 24 hours with or without verteporfin to prepare AI-TEV or control TEV (C-TEV), respectively. When TEVs were analyzed by nanoparticle tracking analysis and cryo-transmission electron microscopy, E.G7-OVA TEVs had a uniform round shape with a diameter of approximately 118 nm (FIGS. 8 and 9). Similar patterns were observed for MOC2 and 4T1 TEVs (FIGS. 10 and 11). TEVs expressed several EV markers such as tumor susceptibility genes 101 (TSG101), CD81, and Alix, but had no negative marker calnexin (FIGS. 12 and 13). Since verteporfin inhibited both autophagy and YAP, it was expected that there was a significant difference in a proteomic cargo between C-TEV and AI-TEV. This result was confirmed by proteomic analysis and indicated in the scatter plot of normalized data (FIG. 14). A total of 2,509 proteins were detected in both samples, and among them, 529 proteins showed a relative increase in AI-TEV and 315 proteins showed an increase in C-TEV.

### Example 3. Confirmation of Tumorigenic Properties Reduced by Verteporfin in TEV

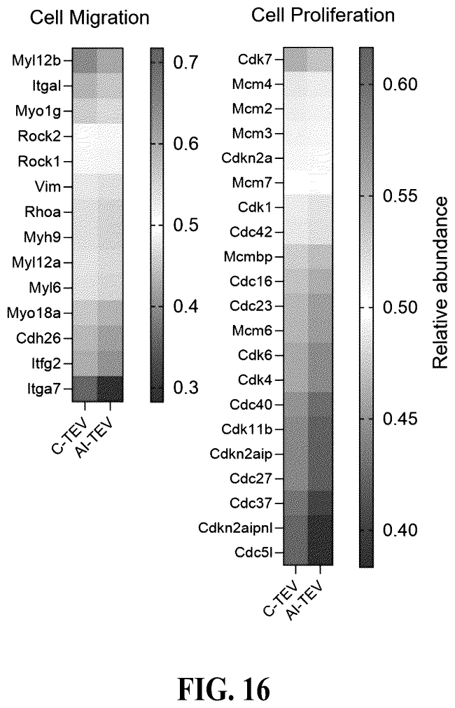

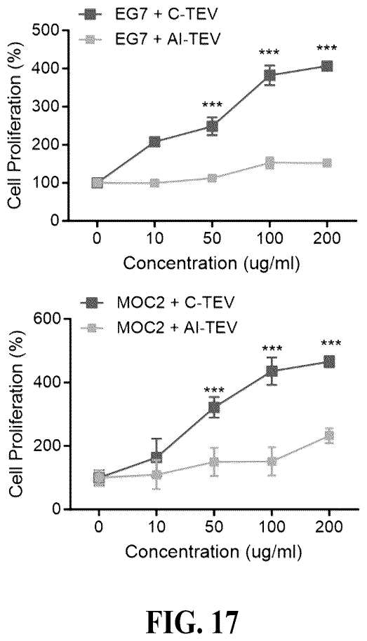

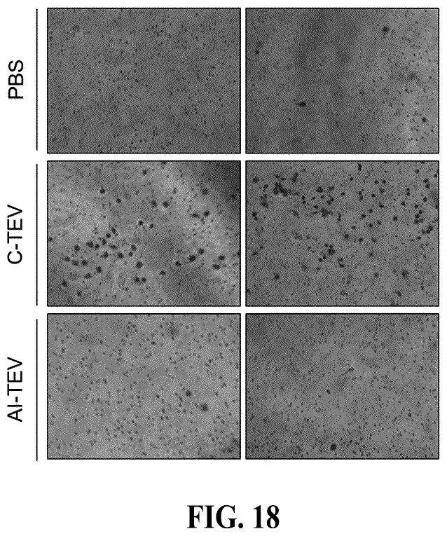

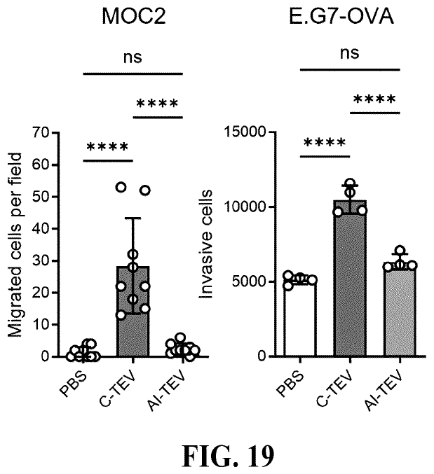

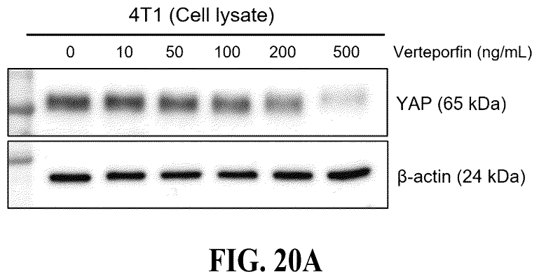

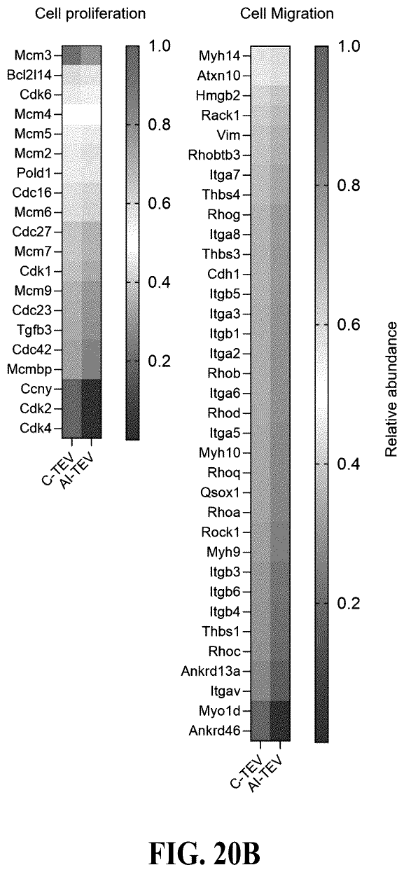

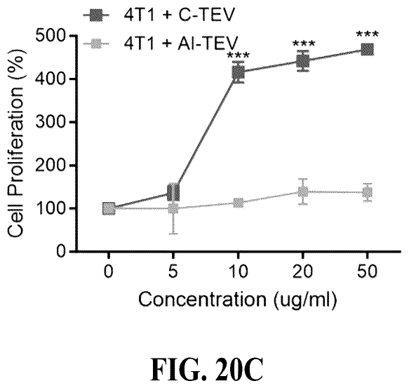

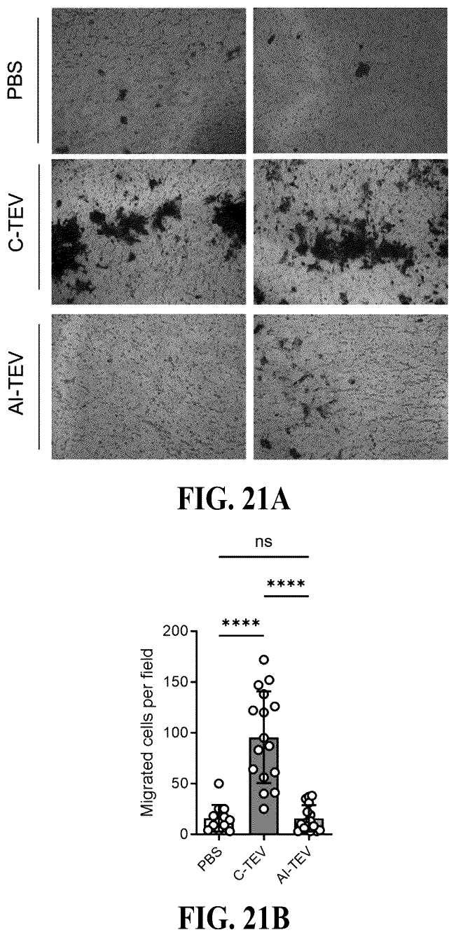

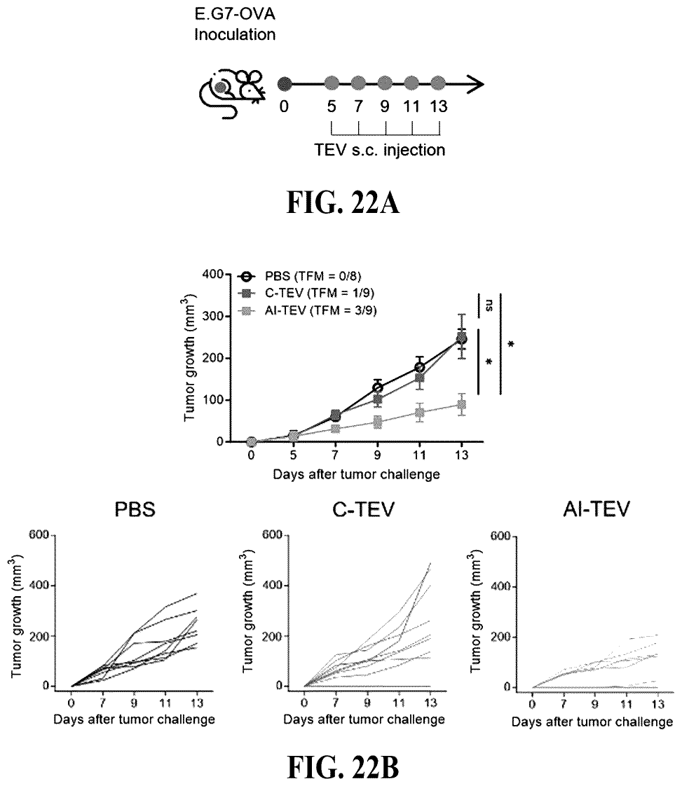

Since verteporfin was known to prevent YAP-TEAD interaction and promote YAP degradation in the cytoplasm, it was confirmed that verteporfin treatment decreased the YAP protein content in E.G7-OVA and MOC2 cells (FIG. 15). As a result, proteomic data analysis of E.G7-OVA TEV showed a significant decrease in most proteins downstream of YAP-TEAD signaling, particularly proteins involved in cell migration and proliferation (FIG. 16). Considering that YAP was a well-known oncogene that promoted cancer cell metastasis and progression, it was demonstrated that AI-TEV delivered much less pro-tumorigenic substances than C-TEV due to YAP inhibition. To further demonstrate the reduced tumor-promoting properties of AI-TEV, cancer cells were cultured with each TEV for 24 hours and observed that C-TEV induced a significant increase in cancer cell proliferation, whereas AI-TEV did not induce the significant increase in cancer cell proliferation (FIG. 17). In addition, the migration ability of TEV-stimulated tumor cells was evaluated using a transwell migration assay. Microscopic examination showed that MOC2 cells cultured with C-TEV exhibited significant migration, whereas cells cultured with AI-TEV exhibited significantly less migration, and there was no significant difference between cells treated with phosphate-buffered saline (PBS) and cells treated with AI-TEV. E.G7-OVA cells were also evaluated using the same protocol, but due to non-adherent characteristics, invasive cells were counted in the lower wells of a transwell chamber rather than on a membrane (FIGS. 18 and 19). Similar results were observed even in 4T1 cells, which indicated verteporfin-mediated YAP inhibition and the resulting differences in protein cargo and proliferation of TEV-treated tumor cells (FIGS. 20A to 20C). Due to the aggressive and metastatic characteristics of 4T1 cells, a matrigel coating was added to the transwell membrane for the invasion assay. Microscopic analysis of TEV-treated 4T1 cells showed significant differences in invasion between C-TEV and AI-TEV-treated cells (FIG. 21A and 21B). Finally, to confirm the attenuation of the tumor-promoting properties of AI-TEV in vivo, the efficacy as a therapeutic vaccine was evaluated in a mouse model. The therapeutic vaccine was another well-studied form of tumor vaccine designed to suppress tumor growth and induce long-term antitumor memory. Among E.G7-OVA tumor-bearing mice injected with TEV or PBS (FIG. 22A), mice injected with AI-TEV showed a higher ability to delay tumor growth compared to other groups, and 3 out of 9 mice were tumor-free (FIG. 22B). None of 9 mice in the C-TEV group were tumor-free, but the remaining 8 mice showed rapid and aggressive tumor growth similar to the PBS group to have no significant difference between the two groups. These results showed that C-TEV could not inhibit tumor growth, while AI-TEV attenuated these properties to exhibit the potential as a vaccine that selectively delivered the desired properties of TEV without tumor-promoting effects.

### Example 4. Confirmation of cGAS-STING Pathway Activation and DC Maturation Promotion Effects of AI-TEV

For TEV to be an effective cancer vaccine, an adjuvant is required to sufficiently stimulate the innate immune system as well as to be free of tumor-promoting aspects. Accordingly, the following experiments were conducted to demonstrate that AI-TEV may induce a strong immune response by serving as both a vaccine and an adjuvant. The DC was an antigen-presenting cell that acted as a mediator between innate immunity and adaptive immunity and was involved in direct activation of CD8+ cytotoxic T cells. Therefore, to evaluate the potential of AI-TEV, it was important to evaluate the ability to activate DC. Since verteporfin was widely used as an autophagy inhibitor, it was predicted that verteporfin would accumulate tumor antigens and various cofactors, especially DAMPs and nucleic acids, in the cytoplasm and then increase their release through TEVs.

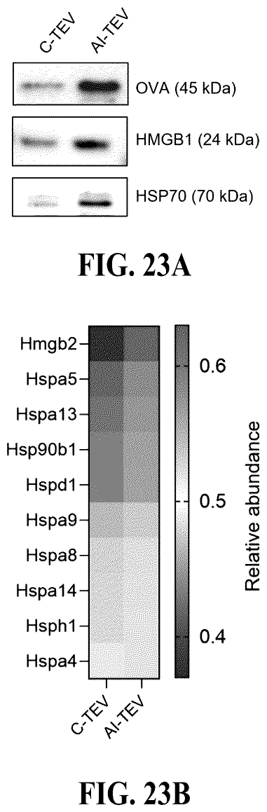

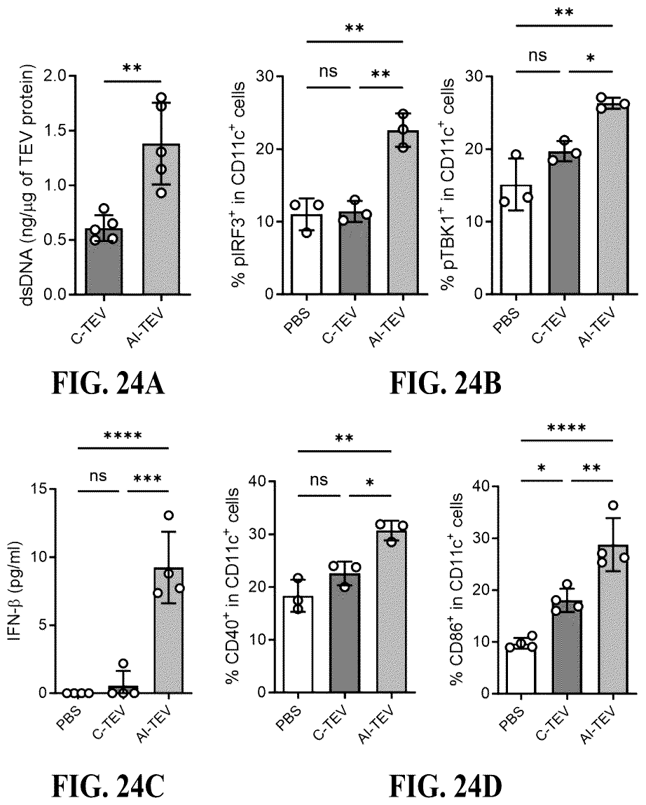

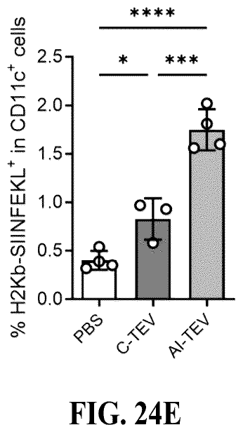

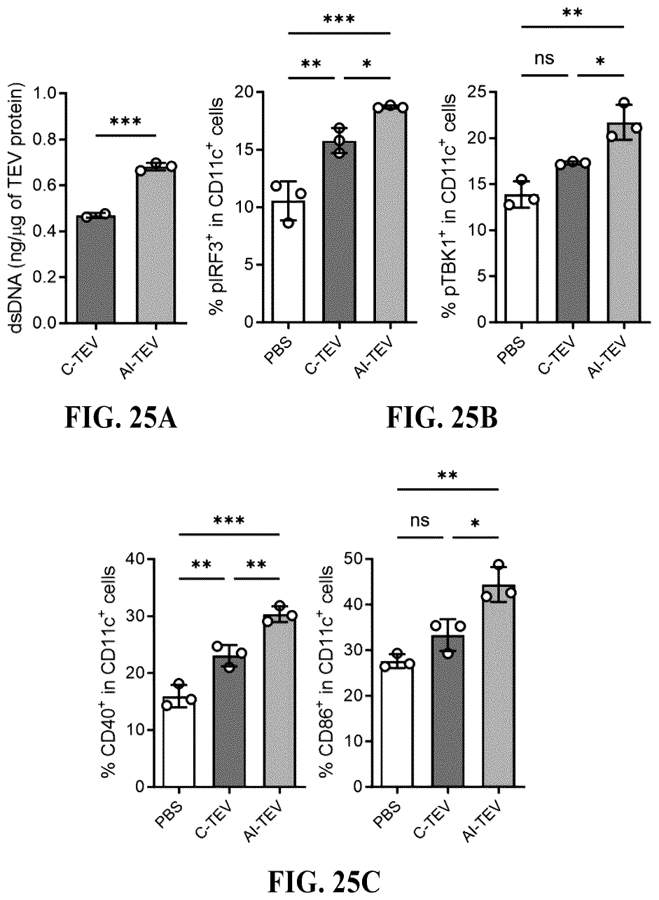

As expected, immunoblotting of E.G7-OVA-derived TEVs showed a significant increase in ovalbumin (OVA) and DAMPs (HSP70 and HMGB1) in AI-TEVs (FIG. 23A). Furthermore, proteomic analysis of TEV cargos showed that most DAMPs were increased in AI-TEVs compared to C-TEVs (FIG. 23B). Previous studies have reported that TEVs carrying dsDNA derived from cancer cells activated the cGAS-STING pathway in DCs. A recent study demonstrated the fact that inhibition of autophagy in leukemic cells increased the accumulation of cytoplasmic dsDNA and then the cytoplasmic dsDNA was subsequently delivered to EVs, so that the cGAS-STING pathway was activated in bone marrow cells. Therefore, the present inventors predicted that inhibition of verteporfin-mediated autophagy would increase the amount of dsDNA in the cytoplasm of tumor cells and AI-TEVs, resulting in enhanced activation of the cGAS-STING pathway in DCs. It was confirmed that AI-TEV carried much more dsDNA than C-TEV using the QuantiFluor dsDNA system, and E.G7-OVA-derived AI-TEV showed almost 3-fold increase in dsDNA compared to C-TEV (FIGS. 24A and 25A). Therefore, it was confirmed that AI-TEV carried large amounts of tumor antigens, DAMPs and dsDNA. Thereafter, the ability of AI-TEV to activate DCs was evaluated by treating bone marrow-derived dendritic cells (BMDCs) with TEVs and analyzing the differences by flow cytometry. Notable markers for the cGAS-STING pathway, phosphorylated interferon regulatory factor 3 (pIRF3) and phosphorylated TANK binding kinase 1 (pTBK1), were increased in DCs treated with AI-TEV compared with other groups (FIGS. 24B and 25B). A significant increase in IFN-β release from these DCs was also observed (FIG. 24C), which showed effective activation of the cGAS-STING pathway. Uptake of DAMPs delivered via TEVs and activation of the cGAS-STING pathway also resulted in an increase in the levels of mature markers for DCs (CD40 and CD86) (FIGS. 24D and 25C). BMDCs cultured with E.G7-OVA-derived TEVs were analyzed for cross-presentation of OVA (SIINFEKL) peptides and showed a highly significant enhancement of DC cross-presentation in the group treated with AI-TEVs (FIG. 24E). Overall, these results demonstrate that AI-TEV has sufficient antigens and adjuvants to induce significant activation of DCs, enhance cross-presentation, and demonstrate the potential to induce a robust and sustained immune response.

### Example 5. Confirmation of Tumor-Specific Immunity Promotion of AI-TEV as Preventive Vaccine

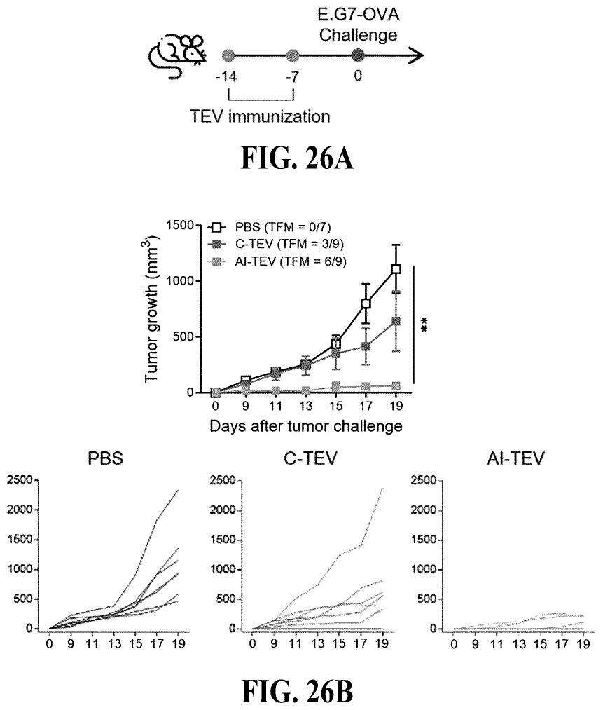

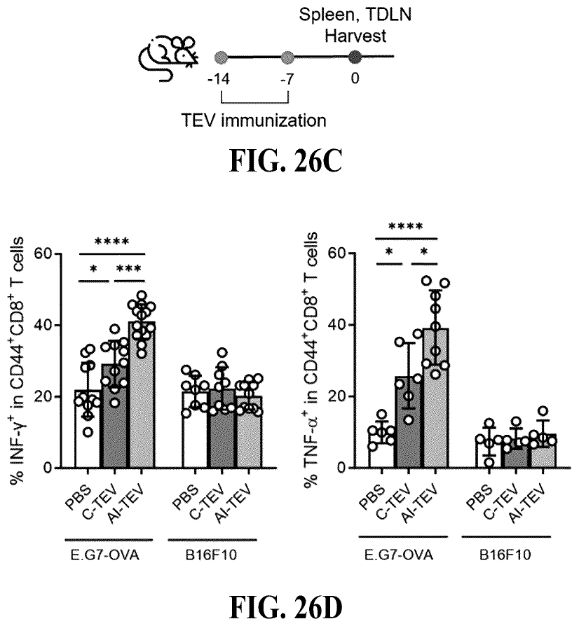

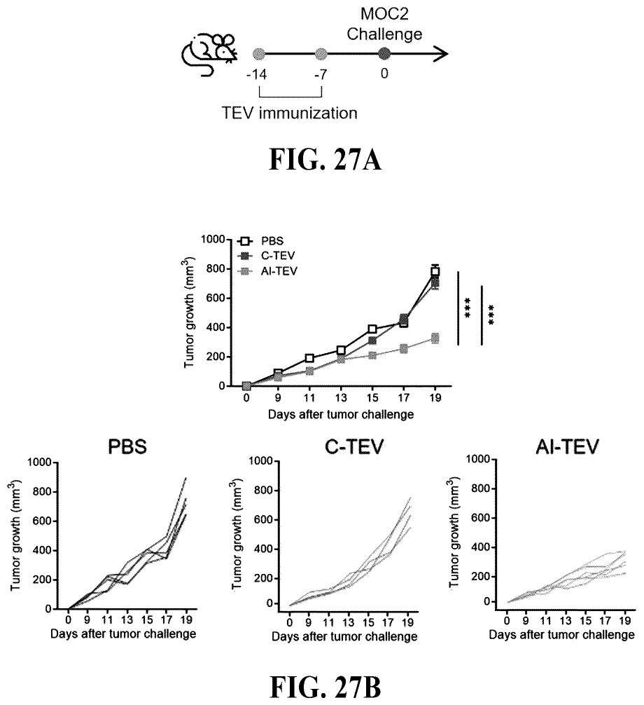

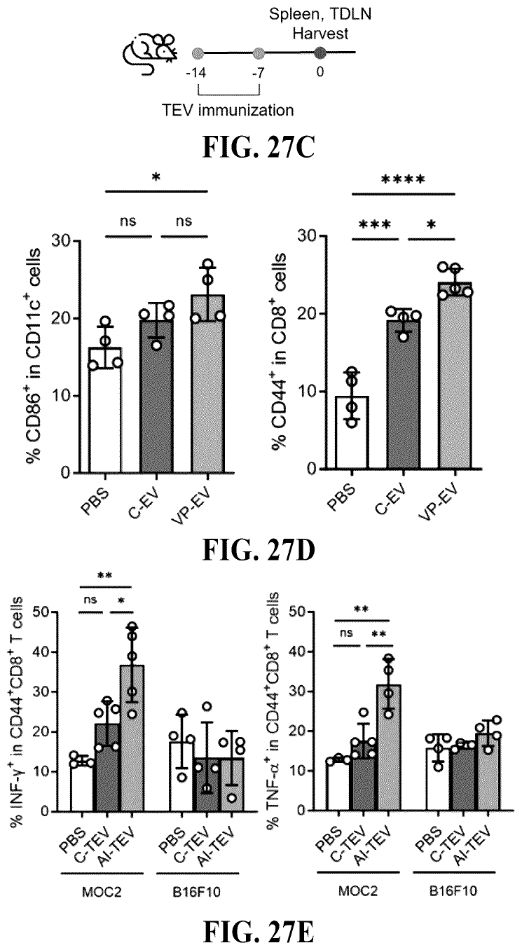

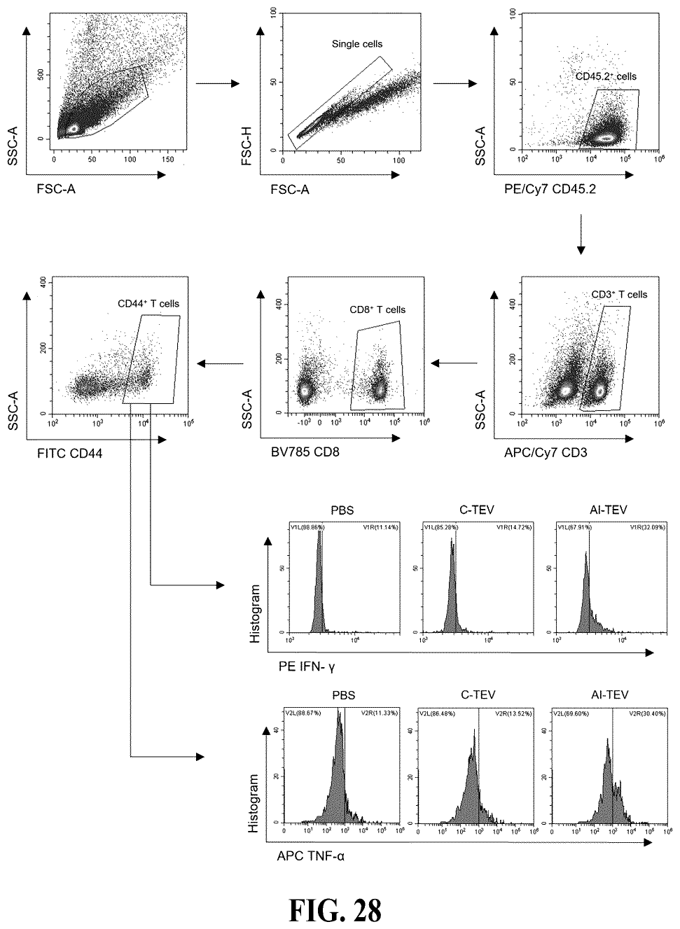

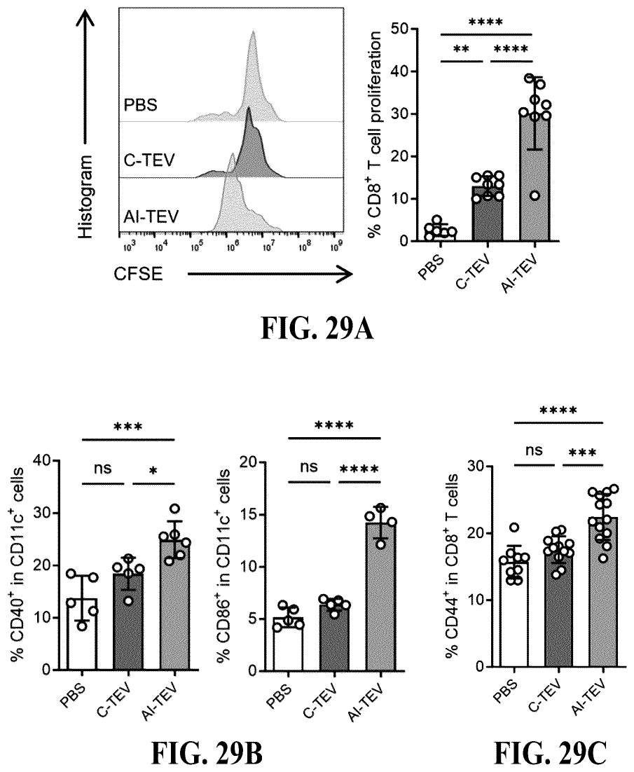

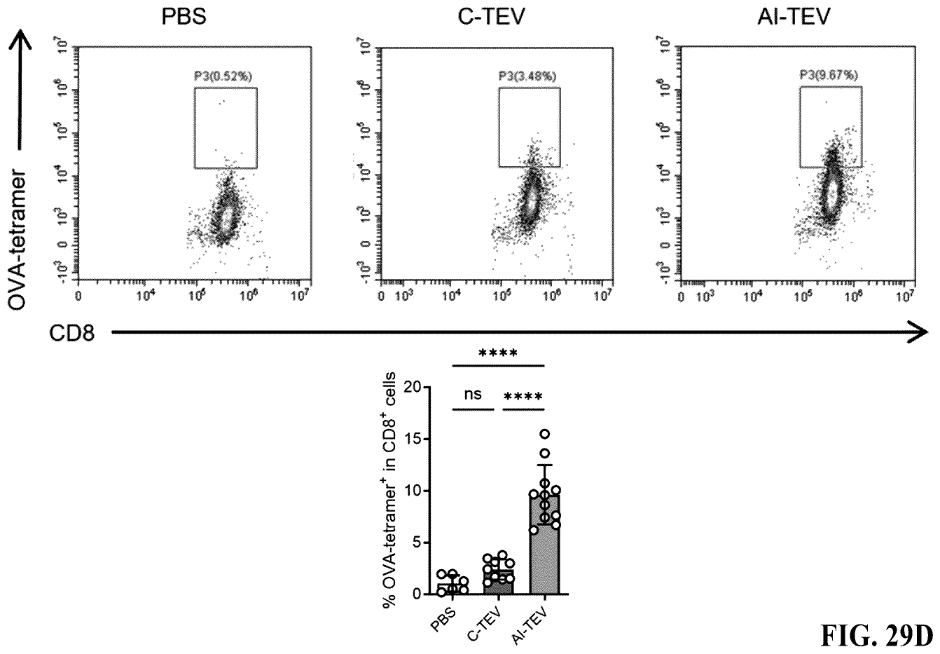

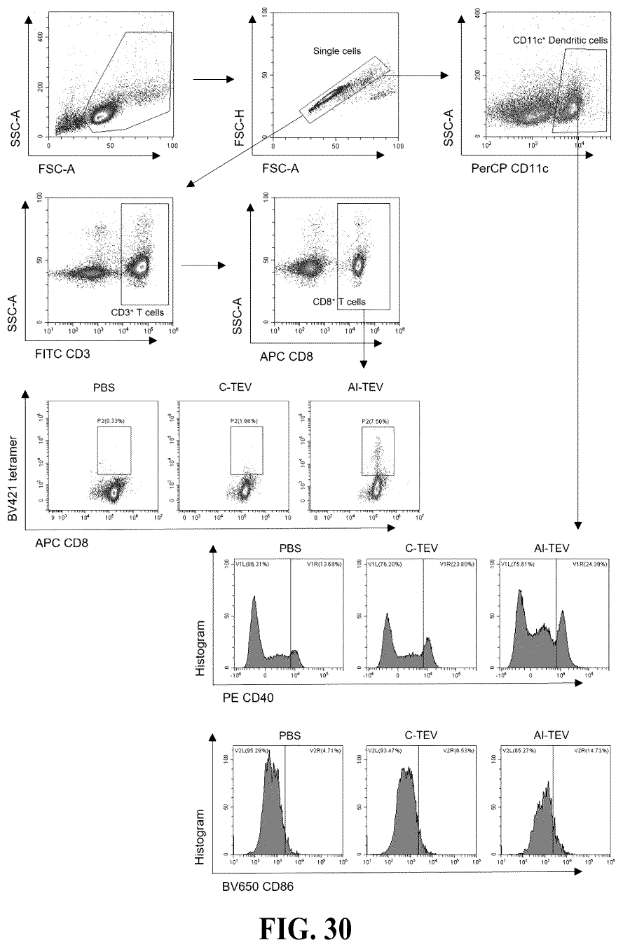

The most important aspect of a cancer vaccine is the ability to prepare the immune system in a tumor-specific manner and prevent the occurrence or recurrence of cancer. The present inventors attempted to confirm the potential of AI-TEV as a preventive vaccine by inoculating E.G7-OVA tumor cells with TEV every week through subcutaneous injection (FIG. 26A). AI-TEV vaccination effectively delayed tumor growth compared to other groups, and more than half of the experimental animals remained tumor-free for almost 3 weeks (FIG. 26B). Delayed tumor growth was also observed in mice injected with C-TEV, which was expected due to the high immunogenicity of E.G7-OVA cells and the fact that C-TEV was already known to carry tumor antigens. Therefore, a somewhat limited preventive effect was expected for C-TEV. However, it was confirmed that this effect was amplified in a tumor-specific manner in AI-TEV. This preventive effect was also demonstrated using MOC2 TEV (FIGS. 27A and 27B). To verify whether AI-TEV induced tumor-specific immunity, spleen and tumor-draining lymph nodes (TDLN) were extracted from mice vaccinated with E.G7-OVA TEV (FIG. 26C). Splenocytes were pulsed ex vivo with two types of irradiated cancer cells (E.G7-OVA or B16F10 as controls). Flow cytometry of splenocytes to determine the percentage of activated effector CD8+ T cells (FIG. 28) showed that the percentage of IFN-γ+CD44+ CD8+ T and TNF-α+ CD44+ CD8+ T cells was significantly higher in splenocytes from AI-vaccinated mice. TEVs were higher than in mice vaccinated with C-TEV and PBS. Importantly, splenocytes pulsed with B16F10 cells, not with individual cancer cells, showed no remarkable difference, which suggested a tumor-specific response of splenocytes (FIG. 26D and FIGS. 27C and 27D) and the ability of AI-TEV to educate splenocytes. To further confirm the tumor-specific immune response, splenocytes of vaccinated mice were labeled with carboxyfluorescein diacetate-succinimidyl ester (CFSE) and cultured with irradiated E.G7-OVA cells for 72 hours. Flow cytometry to confirm the proliferation of CFSE-positive CD8+ T cells showed that CFSE was highly significantly diluted in the AI-TEV group compared with other groups, which suggested a strong tumor-specific response (FIG. 29A). Moreover, mature DCs (CD40+ CD11c+ CD86+ CD11c+) and effector T cells (CD44+ CD8+ T cells) were observed in TDLN cells, which indicated that the ability of DCs to prime CD8+ T cells in lymph nodes was enhanced (FIGS. 29B and 29C, FIG. 27E, and FIG. 30). To further confirm this theory, TDLN cells were stained with MHC tetramer to detect SIINFEKL-specific T cell populations (FIG. 29D). The tetramer analysis showed a significant difference in the OVA-tetramer+ CD8+ T cell population, which indicated an increased proportion of tumor antigen-specific CD8+ T cells in AI-TEV-injected mice. These results show that AI-TEV can be used as a preventive vaccine by inducing tumor-specific immunity.

### Example 6. Confirmation of Long-Term Memory Induction Effect of AI-TEV

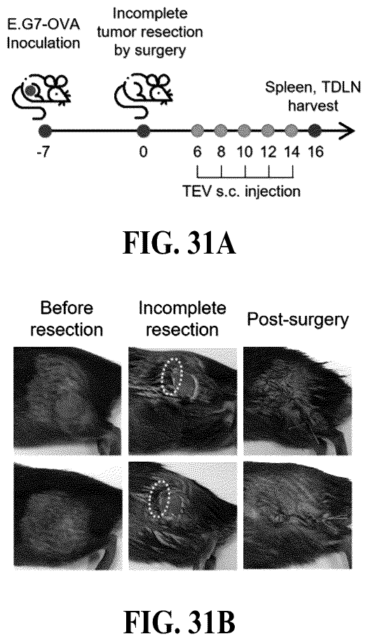

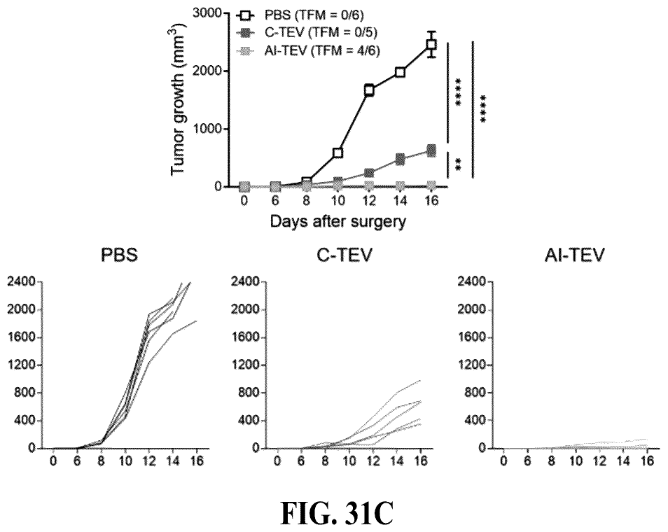

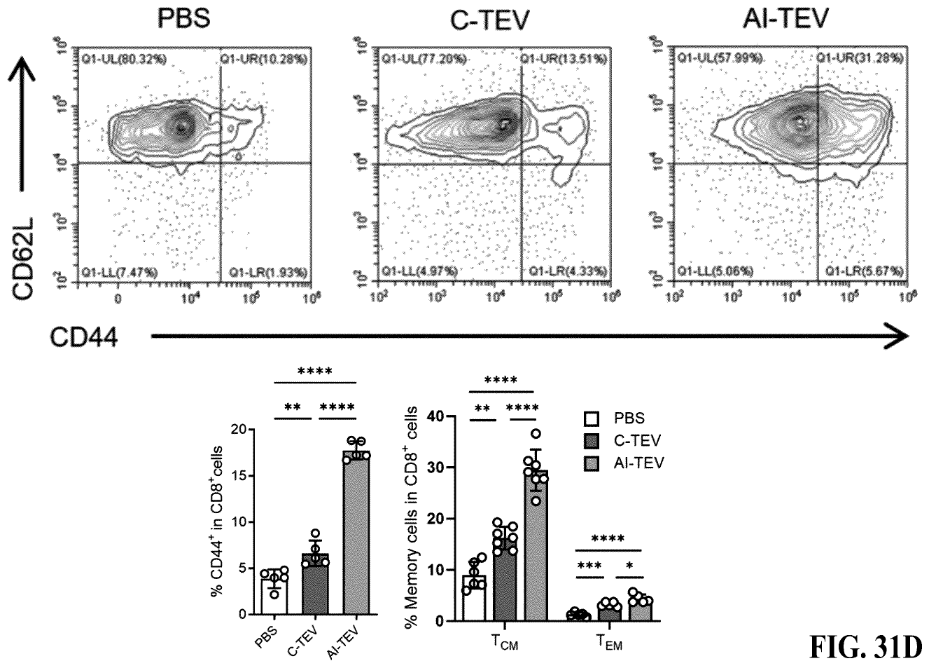

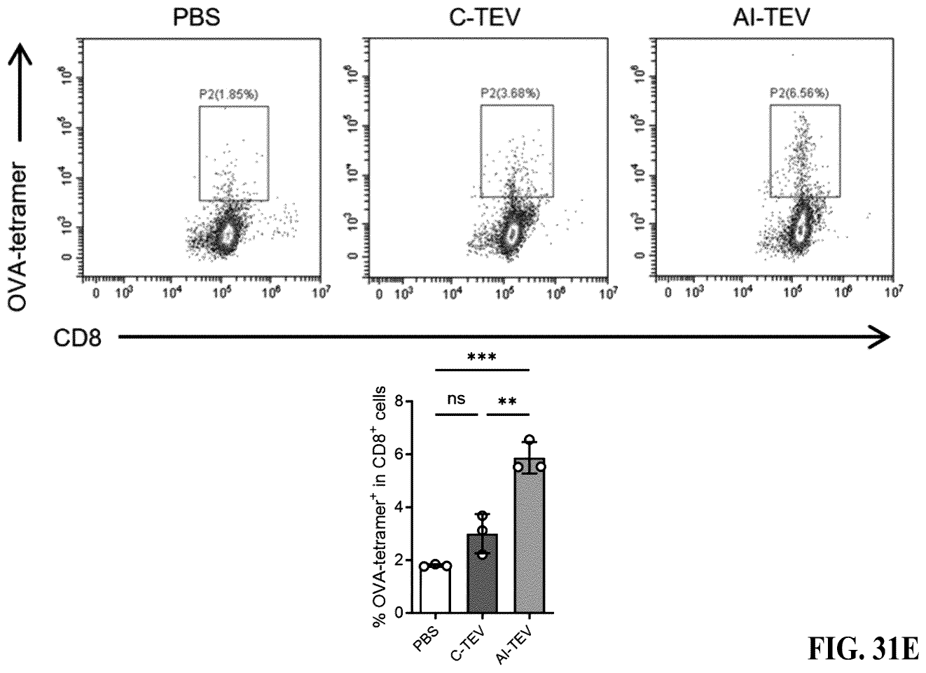

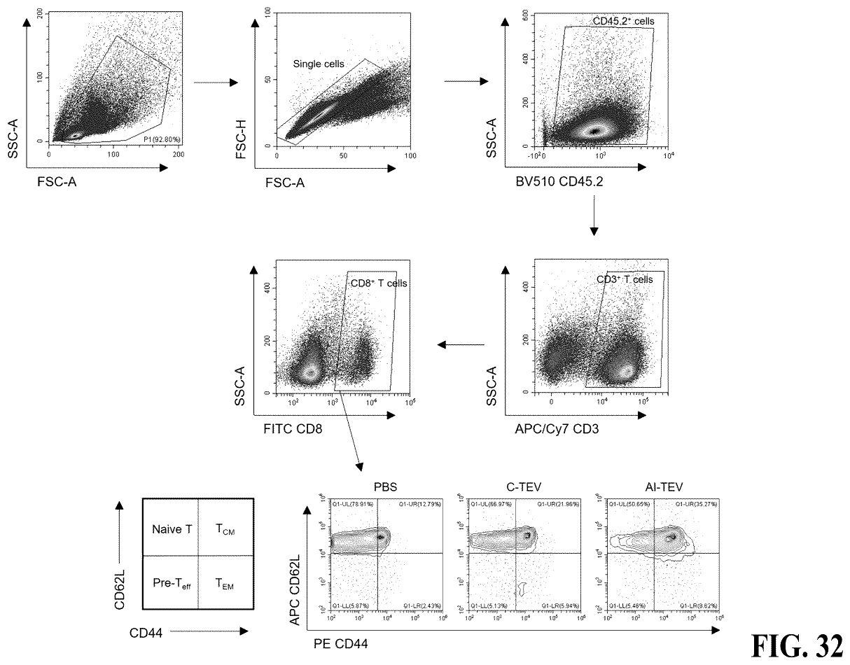

Traditional vaccines are intended to completely prevent the occurrence of target diseases, but all cancers cannot be prevented or predicted in occurrence, so that the mechanism of such vaccines is limited. Therefore, it was aimed to develop a cancer vaccine that may act as an adjuvant therapy usable after primary eradication of tumors and target DCs and educate the immune system to prevent the recurrence or progression of remaining cancers. When initial cancer treatment (surgical resection or chemotherapy) fails to eradicate cancer, such a vaccine should be able to slow or prevent further growth of cancer. The present inventors attempted to confirm the potential of AI-TEV as a postoperative immunotherapy to prevent cancer recurrence by establishing a tumor recurrence mouse model using E.G7-OVA cells (FIG. 31A). After incomplete resection of the primary tumor (approximately 10% mass was maintained) (FIG. 31B), TEV treatment was started on postoperative day 6 (once every 2 days, a total of 5 injections) and tumor recurrence growth was monitored. AI-TEV injection significantly prevented tumor recurrence, and 4 out of 6 mice showed complete tumor regression at the end of the experimental period (day 16) (FIG. 31C). The analysis of the spleen showed a significantly higher proportion of CD44+ CD8+ T cells in the AI-TEV group, CD44hi CD62hi CD8+ T cells (central memory T cells, TCM) was significantly increased, but CD44hi CD62Llo CD8+ T cells (effector memory T cells, TEM) was small, but still significantly increased (FIGS. 31D and 32). In addition, in tetramer analysis of TDLN cells, it was shown that the proportion of OVA tetramer+CD8+ T cells was greatly increased, which indicated that long-term memory induced by AI-TEV was tumor antigen-specific (FIG. 31E). Overall, these results suggest that AI-TEV could be used as an adjuvant therapy to prevent recurrence of remaining cancers by strongly increasing tumor-specific memory from an immune perspective.

### Example 7. Confirmation of Availability of AI-TEV as Customized Cancer Vaccine Platform

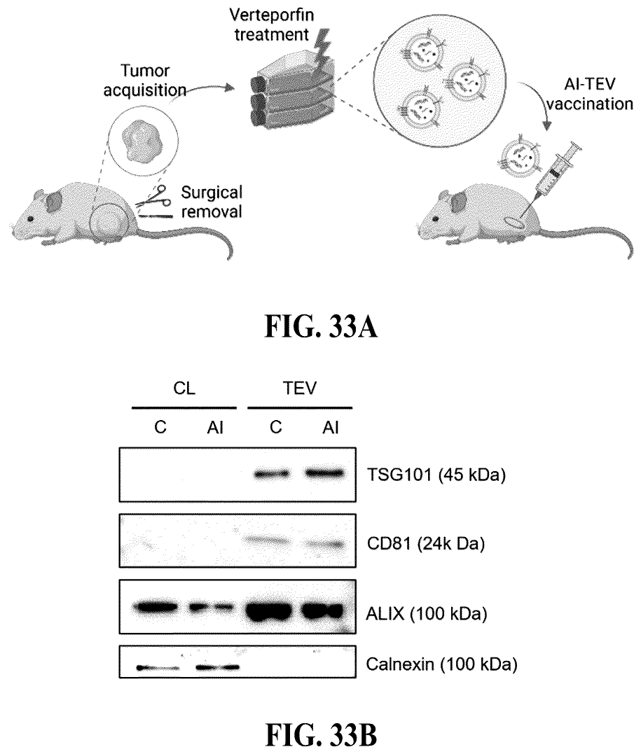

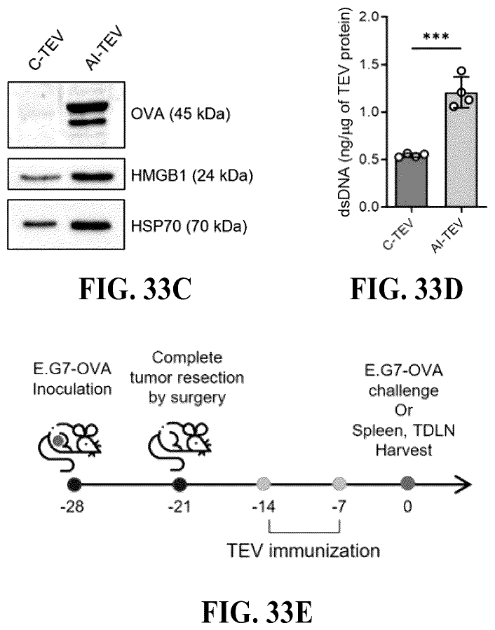

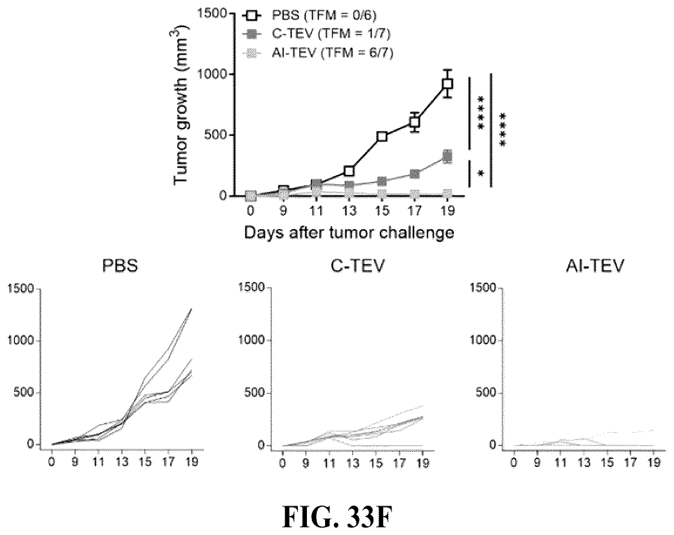

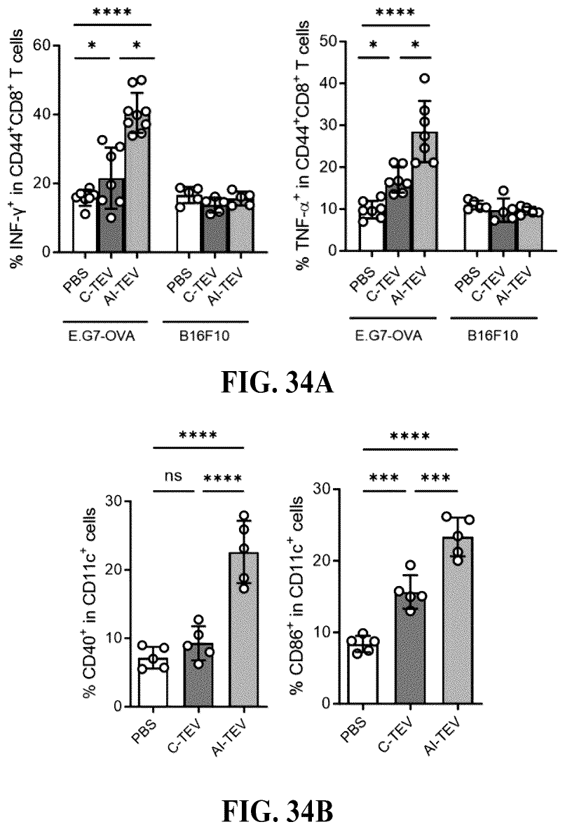

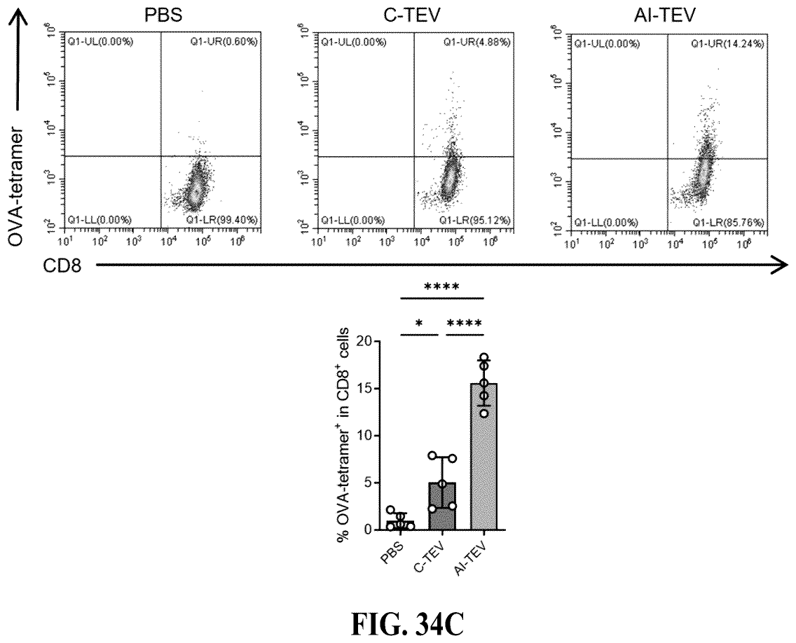

The importance of customized cancer vaccines is more evident because cancer cells are known to exhibit abnormal and mutant behaviors, and the need to trigger customized immune responses to neoantigens is emphasized. Applying autologous tumor cells derived directly from individual patients to isolate AI-TEV as a customized vaccine is a promising therapeutic approach, and in order to confirm this, an experiment was conducted as follows using a customized cancer vaccine model. Tumors were induced in mice by inoculation with E.G7-OVA tumor cells, completely resected after 7 days, and suspended in culture medium. TEVs were isolated from the cultured tumor cells and used for vaccination in the same mice, which were challenged with the same cells or euthanized for analysis (FIG. 33A). Immunoblotting of these TEVs showed the presence of EV markers (FIG. 33B) and an increase in OVA protein and DAMP in AI-TEV compared to C-TEV (FIG. 33C). Quantification of dsDNA cargos showed a greater increase in dsDNA with AI-TEV than with C-TEV (FIG. 33D). These data demonstrate that personalized AI-TEV carries high levels of tumor antigen and adjuvant to be suitable for vaccination. The protective effect of TEV vaccination in a customized cancer vaccine model was demonstrated by re-vaccinating mice with previously challenged E.G7-OVA cells (FIG. 33E). AI-TEV showed dramatic results, which had no tumor in all mice except for one mouse after nearly 3 weeks (FIG. 33F). Further analysis of spleens and TDLN from these vaccinated mice confirmed significant ex vivo activation of tumor-pulsed splenocytes determined by increased proportions of IFN-γ+ and TNF-α+ CD44+ CD8+ T cells (FIG. 34A). As the result of analyzing TDLN cells, it was shown that the numbers of mature DCs (CD40+ CD11c+ and CD86+ CD11c+) and OVA-tetramer+ CD8+ T cells were increased (FIGS. 34B and 34C). These data indicate that personalized AI-TEV may induce potent and tumor-specific immune responses and potentially serve as a tumor vaccine designed at an individual level.

As described above, although the embodiments have been described by the restricted drawings, various modifications and variations can be applied on the basis of the embodiments by those skilled in the art. For example, even if the described techniques are performed in a different order from the described method, and/or components such as a system, a structure, a device, a circuit, and the like described above are coupled or combined in a different form from the described method, or replaced or substituted by other components or equivalents, an appropriate result can be achieved.

Therefore, other implementations, other preparation embodiments, and equivalents to the appended claims fall within the scope of the claims to be described below.

What is claimed is:

1. A tumor-derived vesicle composition prepared through followings:
1) providing a cancer cell line;
2) treating the cancer cell line with verteporfin; and
3) extracting extracellular vesicles from the cancer cell line.

2. The tumor-derived vesicle composition of claim 1, wherein the cancer cell line is at least any one selected from the group consisting of E.G7-OVA, MOC2, and 4T1.

3. The tumor-derived vesicle composition of claim 1, wherein the verteporfin is treated at 10 to 200 ng/mL.

4. The tumor-derived vesicle composition of claim 1, wherein the extracellular vesicles have a diameter of 50 to 200 nm.

5. The tumor-derived vesicle composition of claim 1, wherein the extracellular vesicles are attenuated yet immunogenically potentiated.

6. The tumor-derived vesicle composition of claim 1, wherein the extracellular vesicles overexpress damage-associated molecular patterns (DAMPs).

7. The tumor-derived vesicle composition of claim 6, wherein the damage-associated molecular pattern is at least any one selected from 70 kilodalton heat shock proteins (Hsp70) and high mobility group box 1 (HMGB1).

8. The tumor-derived vesicle composition of claim 1, wherein the extracellular vesicles activate dendritic cells.

9. An anticancer vaccine composition comprising the extracellular vesicles of claim 1 as an active ingredient.

10. The anticancer vaccine composition of claim 9, wherein the anticancer vaccine composition induces tumor-specific immunity.

11. An anticancer immune adjuvant composition comprising the extracellular vesicles of claim 1 as an active ingredient.

12. The anticancer immune adjuvant composition of claim 11, wherein the anticancer immune adjuvant composition induces long-term memory to prevent recurrence of cancer.

13. A method for preparing a tumor-derived vesicle composition, comprising followings:
1) providing a cancer cell line;
2) treating the cancer cell line with verteporfin (VP); and
3) extracting extracellular vesicles from the cancer cell line.

14. The method for preparing the tumor-derived vesicle composition of claim 13, wherein the cancer cell line is at least any one selected from the group consisting of E.G7-OVA, MOC2, and 4T1.

15. The method for preparing the tumor-derived vesicle composition of claim 13, wherein the verteporfin is treated at 10 to 200 ng/ml. in

16. A method for preparing a subject-customized vaccine composition, comprising followings:
1) collecting tumor cells from a subject;
2) treating the tumor cells with verteporfin; and
3) extracting extracellular vesicles from the tumor cells.
